SEQUENCE LISTING (21 sequences)

SEQ ID NO: 1 - protein, 474 aa, Oldenlandia affinis
MVRYLAGAVLLLVVLSVAAAVSGARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWCYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHLWEKFRKAPEGSARKEEAQTQIFKAMSHRVHIDSSIKLIGKLLFGIEKCTEILNAVRPAGQPLVDDWACLRSLVGTFETHCGSLSEYGMRHTRTIANICNAGISEEQMAEAASQACASIP

SEQ ID NO: 2 - protein, 474 aa, Oldenlandia affinis
MVRYLAGAVLLLVVLSVAAAVSGARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWAYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHLWEKFRKAPEGSARKEEAQTQIFKAMSHRVHIDSSIKLIGKLLFGIEKCTEILNAVRPAGQPLVDDWACLRSLVGTFETHCGSLSEYGMRHTRTIANICNAGISEEQMAEAASQACASIP

SEQ ID NO: 3 - protein, 420 aa, Oldenlandia affinis
GTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWAYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHLWEKFRKAPEGSARKEEAQTQIFKAMSHRVHIDSSIKLIGKLLFGIEKCTEILNAVRPAGQPLVDDWACLRSLVGTFETHCGSLSEYGMRHTRTIANICNAGISEEQMAEAASQACASIP

SEQ ID NO: 4 - protein, 297 aa, synthetic construct
GTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWCYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDAD

SEQ ID NO: 5 - protein, 23 aa, synthetic construct
MHHHHHHSSGVDLGTENLYFQSV

SEQ ID NO: 6 - protein, 320 aa, Synthetic construct
MHHHHHHSSGVDLGTENLYFQSVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWCYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDAD

SEQ ID NO: 7 - protein, 462 aa, Viola yedoensis
MGLDVDSLQLPSEAAKFFHNDNSTNDDDSIGTRWAVLIAGSKGYHNYRHQADVCHMYQILRKGGVKDENIIVFMYDDIAYNESNPFPGIIINKPGGENVYKGVPKDYTGEDINNVNFLAAILGNKSAIIGGSGKVLDTSPNDHIFIYYADHGAPGKIGMPSKPYLYADDLVDTLKQKAATGTYKSMVFYVEACNAGSMFEGLLPEGTNIYAMAASNSTEGSWITYCPGTPDFPPEFDVCLGDLWSITFLEDCDAHNLRTETVHQQFELVKKKIAYASTVSQYGDIPISKDSLSVYMGTDPANDNRTFVDENSLRPPLKVIHQHDADLYHIWCKYNMAPEGSSKKIEAQKQLLELMSHRAHVDNSITLIGKLLFGVNKASKVLNTVRPVGQPLVDDWQCLKAMIRTFETHCGSLSEYGMKHTLSFANMCNAGIQKEQLAEAAAQACVTFPSNPYSSLAEGFSA

SEQ ID NO: 8 - protein, 432 aa, Viola yedoensis
GTRWAVLIAGSKGYHNYRHQADVCHMYQILRKGGVKDENIIVFMYDDIAYNESNPFPGIIINKPGGENVYKGVPKDYTGEDINNVNFLAAILGNKSAIIGGSGKVLDTSPNDHIFIYYADHGAPGKIGMPSKPYLYADDLVDTLKQKAATGTYKSMVFYVEACNAGSMFEGLLPEGTNIYAMAASNSTEGSWITYCPGTPDFPPEFDVCLGDLWSITFLEDCDAHNLRTETVHQQFELVKKKIAYASTVSQYGDIPISKDSLSVYMGTDPANDNRTFVDENSLRPPLKVIHQHDADLYHIWCKYNMAPEGSSKKIEAQKQLLELMSHRAHVDNSITLIGKLLFGVNKASKVLNTVRPVGQPLVDDWQCLKAMIRTFETHCGSLSEYGMKHTLSFANMCNAGIQKEQLAEAAAQACVTFPSNPYSSLAEGFSA

SEQ ID NO: 9 - protein, 429 aa, Clitoria ternatea
MVEGTRWAVLVAGSKGYVNYRHQADVCHAYQILKKGGLKDENIIVFMYDDIAYNESNPHPGVIINHPYGSDVYKGVPKDYVGEDINPPNFYAVLLANKSALTGTGSGKVLDSGPNDHVFIYYTDHGGAGVLGMPSKPYIAASDLNDVLKKKHASGTYKSIVFYVESCESGSMFDGLLPEDHNIYVMGASDTGESSWVTYCPLQHPSPPPEYDVCVGDLFSVAWLEDCDVHNLQTETFQQQYEVVKNKTIVALIEDGTHVVQYGDVGLSKQTLFVYMGTDPANDNNTFTDKNSLGTPRKAVSQRDADLIHYWEKYRRAPEGSSRKAEAKKQLREVMAHRMHIDNSVKHIGKLLFGIEKGHKMLNNVRPAGLPVVDDWDCFKTLIRTFETHCGSLSEYGMKHMRSFANLCNAGIRKEQMAEASAQACVSIP

SEQ ID NO: 10 - protein, 426 aa, Clitoria ternatea
GTRWAVLVAGSKGYVNYRHQADVCHAYQILKKGGLKDENIIVFMYDDIAYNESNPHPGVIINHPYGSDVYKGVPKDYVGEDINPPNFYAVLLANKSALTGTGSGKVLDSGPNDHVFIYYTDHGGAGVLGMPSKPYIAASDLNDVLKKKHASGTYKSIVFYVESCESGSMFDGLLPEDHNIYVMGASDTGESSWVTYCPLQHPSPPPEYDVCVGDLFSVAWLEDCDVHNLQTETFQQQYEVVKNKTIVALIEDGTHVVQYGDVGLSKQTLFVYMGTDPANDNNTFTDKNSLGTPRKAVSQRDADLIHYWEKYRRAPEGSSRKAEAKKQLREVMAHRMHIDNSVKHIGKLLFGIEKGHKMLNNVRPAGLPVVDDWDCFKTLIRTFETHCGSLSEYGMKHMRSFANLCNAGIRKEQMAEASAQACVSIP

SEQ ID NO: 11 - protein, 456 aa, Clitoria ternatea
MARLNPQKEWDSVIRLPTEPVDADTDEVGTRWAVLVAGSNGYENYRHQADVCHAYQLLIKGGLKEENIVVFMYDDIAWHELNPRPGVIINNPRGEDVYAGVPKDYTGEDVTAENLFAVILGDRSKVKGGSGKVINSKPEDRIFIFYSDHGGPGVLGMPNEQILYAMDFIDVLKKKHASGGYREMVIYVEACESGSLFEGIMPKDLNVFVTTASNAQENSWGTYCPGTEPSPPPEYTTCLGDLYSVAWMEDSESHNLRRETVNQQYRSVKERTSNFKDYAMGSHVMQYGDTNITAEKLYLFQGFDPATVNLPPHNGRIEAKMEVVHQRDAELLFMWQMYQRSNHLLGKKTHILKQIAETVKHRNHLDGSVELIGVLLYGPGKGSPVLQSVRDPGLPLVDNWACLKSMVRVFESHCGSLTQYGMKHMRAFANICNSGVSESSMEEACMVACGGHDAGH

SEQ ID NO: 12 - protein, 428 aa, Clitoria ternatea
GTRWAVLVAGSNGYENYRHQADVCHAYQLLIKGGLKEENIVVFMYDDIAWHELNPRPGVIINNPRGEDVYAGVPKDYTGEDVTAENLFAVILGDRSKVKGGSGKVINSKPEDRIFIFYSDHGGPGVLGMPNEQILYAMDFIDVLKKKHASGGYREMVIYVEACESGSLFEGIMPKDLNVFVTTASNAQENSWGTYCPGTEPSPPPEYTTCLGDLYSVAWMEDSESHNLRRETVNQQYRSVKERTSNFKDYAMGSHVMQYGDTNITAEKLYLFQGFDPATVNLPPHNGRIEAKMEVVHQRDAELLFMWQMYQRSNHLLGKKTHILKQIAETVKHRNHLDGSVELIGVLLYGPGKGSPVLQSVRDPGLPLVDNWACLKSMVRVFESHCGSLTQYGMKHMRAFANICNSGVSESSMEEACMVACGGHDAGH

SEQ ID NO: 13 - protein, 450 aa, Viola canadensis
MGRDITGDGFLRLPSHGNGNSGDVDSKAGTKWAVLIAGSKGYQNYRHQADVCHAYQILRKGGVKDENIIVFMYDDIAYDIRNPYPGTIINSPDKKDVYKGVPKDYTGEDVNVQNFLAVILGNKTALTGGSGKVLDTRPNDHIFIYYTDHGYPGVLGMPTEPYLYANDLIDTLKKKHALGTYEGLVFYVEACESASIFEGLLPDGLNIYVSTAAKAGEGSWVAYCPSQEPPVPAEYGTCVGDLYSVTWMEDSDVYNLRTQTLHQQYELVKNKIAYASTVSQFGDFPISKDSLFEYMGTDPANEKRQYEDEEKSSSPHVGAVHQREADLHHFWDKYQKASEGSRNKVDARKQLVEVMLHRMHVDDSIESIAKLLFGSGAKASEMMNTIRPPGQPLVSDWDCLKTMVRTFETHCGSLSEYGMKYTRFLANICNSGIQKEKMGEASAQVCLNFP

SEQ ID NO: 14 - protein, 422 aa, Viola canadensis
GTKWAVLIAGSKGYQNYRHQADVCHAYQILRKGGVKDENIIVFMYDDIAYDIRNPYPGTIINSPDKKDVYKGVPKDYTGEDVNVQNFLAVILGNKTALTGGSGKVLDTRPNDHIFIYYTDHGYPGVLGMPTEPYLYANDLIDTLKKKHALGTYEGLVFYVEACESASIFEGLLPDGLNIYVSTAAKAGEGSWVAYCPSQEPPVPAEYGTCVGDLYSVTWMEDSDVYNLRTQTLHQQYELVKNKIAYASTVSQFGDFPISKDSLFEYMGTDPANEKRQYEDEEKSSSPHVGAVHQREADLHHFWDKYQKASEGSRNKVDARKQLVEVMLHRMHVDDSIESIAKLLFGSGAKASEMMNTIRPPGQPLVSDWDCLKTMVRTFETHCGSLSEYGMKYTRFLANICNSGIQKEKMGEASAQVCLNFP

SEQ ID NO: 15 - protein, 450 aa, Viola Uliginosa
MGLDVDPLRLPSEAAKFFHNDNSTNDDDSIGTRWAVLIAGSKDYHNYRHQADVCHMYQILRKGGVKDENIIVFMYDDIAYNESNPHPGIIINKPGGEDVYKGVPKDYTGEDVNNINFLAAILGNKSAIIGGSGKVLDTSPNDHIFIYYTDHGAPGKIGMPSKPYLYADDLVDTLKQKAATGTYKSMVFYVEACNAGSMFEGLLPEGTNIYAMAASNSTEGSWITYCPGATPDFPPEYDICLGDLWSITFLEDCDAHNLRTETVHQQFELVKKNIAYASTVSQYGDIPISKDSLSVYMGTDPANDNRTFVDENSLKPPLKVIHQRDADLYHLWYKYNKAPEGSSKKIEAQKQLLELMSHRAHVDNSITLIGKLLFGVDKASKVLNTVRPVGQPLVDDWQCLKAMIRTFETHCGSLSEYGMKHTLSFANMCNAGIQKEQLAEAAAQACVTFP

SEQ ID NO: 16 - protein, 420 aa, Viola Uliginosa
GTRWAVLIAGSKDYHNYRHQADVCHMYQILRKGGVKDENIIVFMYDDIAYNESNPHPGIIINKPGGEDVYKGVPKDYTGEDVNNINFLAAILGNKSAIIGGSGKVLDTSPNDHIFIYYTDHGAPGKIGMPSKPYLYADDLVDTLKQKAATGTYKSMVFYVEACNAGSMFEGLLPEGTNIYAMAASNSTEGSWITYCPGATPDFPPEYDICLGDLWSITFLEDCDAHNLRTETVHQQFELVKKNIAYASTVSQYGDIPISKDSLSVYMGTDPANDNRTFVDENSLKPPLKVIHQRDADLYHLWYKYNKAPEGSSKKIEAQKQLLELMSHRAHVDNSITLIGKLLFGVDKASKVLNTVRPVGQPLVDDWQCLKAMIRTFETHCGSLSEYGMKHTLSFANMCNAGIQKEQLAEAAAQACVTFP

SEQ ID NO: 17 - protein, 15 aa, synthetic construct
GLPVSTKPVATRNAL

SEQ ID NO: 18 - protein, 7 aa, synthetic construct
PIEYNAL

SEQ ID NO: 19 - protein, 6 aa, synthetic construct
GIKSIP

SEQ ID NO: 20 - protein, 11 aa, synthetic construct
PIEYNGIKSIP

SEQ ID NO: 21 - protein, 8 aa, synthetic construct
GIGGIYRK

(19) United States
(12) Patent Application Publication
(10) Pub. No.: US 2025/0283065 A1
(43) Pub. Date: Sep. 11, 2025

(54) TRUNCATED POLYPEPTIDES HAVING PROTEIN LIGASE ACTIVITY AND METHODS OF PRODUCTION THEREOF

(71) Applicant: NANYANG TECHNOLOGICAL UNIVERSITY, Singapore (SG)

(72) Inventors: Yee Hwa WONG, Singapore (SG); Niying CHUA, Singapore (SG); Julien LESCAR, Singapore (SG); Abbas EL SAHILI, Singapore (SG)

(21) Appl. No.: 18/858,706

(22) PCT Filed: Apr. 24, 2023
(86) PCT No.: PCT/SG2023/050281

§ 371 Date: Oct. 21, 2024

(30) Foreign Application Priority Data
Apr. 22, 2022 (SG) 10202204267Y

(51) Int. Cl. C12N 9/50

(52) U.S. Cl. CPC C12N 9/50; C12Y 304/22

(57) ABSTRACT

Various embodiments relate generally to the field of enzyme technology and specifically relate to polypeptides having Asx-specific protein ligase and cyclase activity and to nucleic acids encoding those as well as methods of the manufacture of said enzymes, more particularly methods of producing stable and constitutively active protein ligases.

### CROSS-REFERENCE TO RELATED APPLICATION

This application claims the benefit of priority of Singapore patent application Ser. No. 10202204267Y filed 22 Apr. 2022, the content of which being hereby incorporated by reference in its entirety for all purposes.

### TECHNICAL FIELD

Various embodiments relate generally to the field of enzyme technology and specifically relate to polypeptides having Asx-specific protein ligase and cyclase activity and to nucleic acids encoding those as well as methods of the manufacture of said enzymes, more particularly methods of producing stable and constitutively active protein ligases.

### BACKGROUND

Enzyme-mediated peptide ligation [1,2] has been exploited for a wide range of applications such as protein/peptide ligation, cyclization and labelling, protein thioester formation [3], protein conjugation to various moieties such as PEG, lipids or fluorescent probes, live-cell surface labelling [4], nanobody conjugation [5] and antibody-drug conjugation [6-11]. Since its discovery, sortase A has been a popular choice to perform protein conjugation [12], but a significant amount of enzyme is required often approaching 1:1 molar ratio with the target protein. A rather large LPXTG tag must be genetically added to the target protein and the reaction catalysed by sortase A is reversible.

Asparaginyl endopeptidases (AEPs) and Peptide Asparaginyl Ligases (PALs) were discovered in cyclotide-producing plants and both enzymes belong to the cysteine protease family C13. AEPs hydrolyse the Asx-Xaa peptide bond (Asx is Asn or Asp) at the P1 position of the polypeptide substrate [13,14]. In contrast, PALs catalyse peptide bond formation. The discovery of hyperactive PALs such as butelase-1 or VyPAL2 and the engineering of the single mutant OaAEP1b-C247A [16,17] has opened possibilities not envisioned before in the field of bioconjugation. Thus, their discovery has attracted intense research activities to facilitate the usage of PALs for various applications in biotechnology and medicine [1,2].

So far, all PALs reported were expressed recombinantly as zymogens and their enzymatically active isoforms were only obtained following incubation at low pH [17,21,24]. Significant heterogeneity is introduced during this low pH activation stage due to the presence of several closely spaced cleavage sites in the proenzyme and the various isoforms are subsequently difficult to separate via chromatography. Thus, the auto-activation process yields a mixture of heterogeneous activated forms of ligases due to the presence of multiple accessible activation sites at both the N and C termini of the proenzyme. This heterogeneity could be an issue for various industrial applications from a manufacturing and quality control perspective.

Therefore, there is a need in the art for providing protein ligase constructs expressed in an enzymatically active isoform that does not require acid-activation, whereby the expression and subsequent purification of said protein ligases facilitate manufacturing and streamline their utility as molecular tools for ligation and cyclization. In particular, it would be helpful in providing a constitutively active form of protein ligases that retains a level of catalytic activity similar to the acid-activated counterpart, and methods capable of producing such protein ligases.

### SUMMARY

Various embodiments meet this need by in one aspect providing a polypeptide having protein ligase activity, comprising:
(i) an amino acid sequence as set forth in SEQ ID NO: 3 (OaAEP1b C247A core domain+linker+cap domain);
(ii) an amino acid sequence that shares at least 55, preferably at least 60, preferably at least 70, more preferably at least 80, most preferably at least 90% sequence identity with the amino acid sequence of (i) over its entire length; or
(iii) an amino acid sequence that shares at least 70, preferably at least 80, more preferably at least 90, most preferably at least 95% sequence homology with the amino acid sequence of (i) over its entire length,
   wherein the amino acid sequence of (i)-(iii) comprises a C-terminal truncation after amino acid position 351, wherein position numbering is in accordance with SEQ ID NO:1 (OaAEP1b).

In various embodiments, the polypeptide comprises amino acid residue D at the positions corresponding to positions 349 and 351 of SEQ ID NO: 1.

In various embodiments, the polypeptide comprises:
a) amino acid residue A at the position corresponding to position 350 of SEQ ID NO:1; and/or
b) amino acid residue R or H at the position corresponding to position 348 of SEQ ID NO: 1; and/or
c) amino acid residue Q at the position corresponding to position 347 of SEQ ID NO:1.

In various embodiments, the polypeptide comprises:
a) amino acid residue A or V at the position corresponding to position 344 of SEQ ID NO: 1; and/or
b) amino acid residue V or I at the position corresponding to position 345 of SEQ ID NO:1; and/or
c) amino acid residue V, N, H or S at the position corresponding to position 346 of SEQ ID NO: 1; and/or
d) amino acid residue D at the position corresponding to position 351 of SEQ ID NO:1.

In various embodiments, the polypeptide comprises the amino acid sequence set forth in SEQ ID NO:3 (OaAEP1b C247A core domain+linker+cap domain) comprising a C-terminal truncation after amino acid position 351.

In various embodiments, the C-terminal truncation starts at an amino acid position within the first N-terminal helix of the cap domain of the amino acid sequence.

In various embodiments, the polypeptide comprises an amino acid sequence as set forth in SEQ ID NO: 4 (OaAEP1b-C247A-4351), wherein the amino acid residue at the position corresponding to position 351 of SEQ ID NO:1 is the C-terminus of the polypeptide.

In various embodiments, the polypeptide further comprises a His-tag at the N-terminal of the amino acid sequence.

In various embodiments, the polypeptide is a constitutively active protein ligase.

In various embodiments, the polypeptide is a recombinant polypeptide having protein ligase activity, more particularly a recombinantly expressed polypeptide.

In another aspect, there is provided a nucleic acid molecule encoding the polypeptide as disclosed herein.

In another aspect, there is provided a vector comprising the nucleic acid molecule disclosed herein.

In various embodiments, the vector further comprises regulatory elements for controlling expression of said nucleic acid molecule.

In another aspect, there is provided a host cell comprising the nucleic acid molecule disclosed herein or the vector disclosed herein, wherein the host cell is a bacteria cell, preferably an E. coli cell, or an insect cell, preferably an Sf9 cell, or a mammalian cell, preferably a Expi293 cell or a ExpiCHO cell.

In various embodiments, the host cell in an E. coli cell.

In another aspect, there is provided a method for producing a polypeptide having protein ligase activity, comprising: culturing a host cell comprising a nucleic acid molecule encoding the polypeptide disclosed herein under conditions that allows expression of the polypeptide, isolating the polypeptide from the host cell or culture medium, and purifying the polypeptide to obtain the polypeptide having protein ligase activity.

In various embodiments, said nucleic acid molecule is comprised in a vector, preferably an expression vector.

In various embodiments, said vector further comprises regulatory elements for controlling expression of said nucleic acid molecule.

In various embodiments, the host cell is a bacteria cell, preferably an E. coli cell, or an insect cell, preferably an Sf9 cell, or a mammalian cell, preferably a Expi293 cell or a ExpiCHO cell.

In various embodiments, the polypeptide having protein ligase activity is constitutively active.

In various embodiments, the method does not comprise an acid-activation step.

In various embodiments, the method further comprises lysing the host cells and isolating the expressed polypeptide from the lysed cells.

In various embodiments, the polypeptide is expressed as inclusion bodies.

In various embodiments, the method further comprises solubilizing the isolated polypeptide.

In various embodiments, the method further comprises refolding the isolated polypeptide to obtain the polypeptide having protein ligase activity.

All embodiments described above in the context of the polypeptide and concerning the nucleic acid molecule, vector and host cell as such are similarly applicable to the method of the various embodiments.

### BRIEF DESCRIPTION OF THE DRAWINGS

Various embodiments will be better understood with reference to the detailed description when considered in conjunction with the non-limiting examples and the accompanying drawings.

FIG. 1 illustrates the OaAEP1b core-cap domain interface: (A) Structure of OaAEP1b in its zymogen form with residues involved in the interaction between cap and core domains represented by ball-and-sticks; and (B) Electrostatic surface map of the core-cap domain at pH 4.5 and 6.5, respectively, highlighting the electrostatic repulsion between both domains at pH values routinely used for acid-activation of the zymogen. The electrostatic maps were calculated using the APBS server (https://server.poissonboltzmann.org) and visualised using pyMol (Schrodinger inc.).

FIG. 2 illustrates the design of truncated constructs of OaAEP1b: (A) Schematic view of the four constructs of OaAEP1b-C247A that were subjected to expression tests in E. coli; and (B) 3D structure of OaAEP1b-C247A in its proenzyme form (PDB access code: 5H01). Residues 326-342 from the linker region are flexible and could not be traced in the electron density map. The truncation sites introduced to obtain a constitutively active peptide ligase are indicated.

FIG. 3 shows: (A) the annotated amino acid sequence of OaAEP1b-C247A. The amino-acid sequence of the OaAEP1b-C247A proenzyme. The—line indicates the C-terminus of the amino acid sequence of the respective construct. Secondary structure elements are labelled and shown above the sequence. The stretch of amino acids underlined belongs to the signal peptide region, which is removed during the proenzyme maturation, with G55 becoming the N-terminus of the mature enzyme. In contrast, the residue at the C-terminal residue of the purified proenzyme is P474, boldly underlined. The two catalytic residues Cys217 and His175, as well as the gatekeeper residue Cys247 are highlighted in the amino-acid sequence; and (B) sequence alignment of the linker and cap domain of various PALs (i.e. corresponding to amino acid residues at positions corresponding to positions 325-361 of SEQ ID NO: 1), indicating amino acid residue at position 351, and the given or corresponding residues of the α6-helix in OaAEP1b-C247A. The position numbering is in accordance with SEQ ID NO:1.

FIG. 4 shows a schematic view of the dialysis refolding protocol used to purify the constitutively active OaAEP1b-C247A-4351 enzyme. The target OaAEP1b-C247A-A351 protein was expressed as bacterial inclusion bodies and resolubilized with a buffer containing 8M urea. Protein refolding was performed via stepwise removal of urea through dialysis against buffers 2 and 3 for 5-8 hours and 14-16 hours, respectively. Subsequent purification of refolded protein was done by immobilized metal chelating chromatography (IMAC) and Size-exclusion chromatography (SEC). The composition of buffers used for purification is indicated.

FIG. 5 shows the expression in E. coli, refolding, and purification of OaAEP1b-C247A-A351: (A) The left panel shows a 12% SDS PAGE analysis with Coomassie blue staining of the protein at various purification steps, while the right panel shows a western blot of the same gel with a commercial anti-His antibody. A large quantity of expressed protein was observed in the insoluble fraction. The protein was resolubilized and subjected to dialysis. The refolded protein was purified using metal ion affinity chromatography followed by size exclusion chromatography to get a pure homogeneous enzyme that elutes as a monomer; (B) The metal affinity chromatogram shows the protein elution (mAu line curve peak at about 140 ml elution volume) following an increasing amount of imidazole buffer to the column (% B lines); and (C) Gel filtration of the OaAEP1b-C247A-A351 enzyme shows that the enzyme elutes as a monomeric species.

FIG. 6 shows the purified OaAEP1b-C247A-A351 cyclization activity assay: (A) Schematic representation of the cyclization reaction of a linear substrate (“LS”) in the presence of the constitutively active purified PAL; and (B) MALDI TOF mass spectra of the reaction mixture following a series of incubation time points of the linear substrate with the purified OaAEP1b-C247A-A351 revealing only the presence of the cyclized peptide (“CP”) after twelve minutes of incubation time.

FIG. 7 shows the FRET ligation activity assay of OaAEP1b-C247A-A351 and comparison with the acid-activated enzyme: (A) Schematic representation of the ligation reaction of two peptides containing a FRET donor and acceptor, EDANS and DABSYL. Upon ligation, the acceptor molecule, DABSYL, comes in proximity with EDANS, resulting in a quenching of the EDANS emission (λₑₘ=490 nm); and (B) RFU values for the acid-activated OaAEP1b-C247A enzyme were compared with those obtained from the purified truncated OaAEP1b-C247A-Δ351 enzyme. (C) Vₘₐₓ (RFU/sec) and Kₘ (μM) Michaelis values were deduced from two experimental repeats.

FIG. 8 shows active site titration of OaAEP1b-C247A-Δ351: (A) OaAEP1b-C247A-Δ351 activity was measured by FRET ligation assay in presence at increasing concentrations of ac-YVAD-cmk covalent inhibitor [34]. The measured concentration of the enzyme is 280 nM. In order from the top (closest to 30000 rfu at 14 mins) to bottom (closest to 0 rfu at 14 mins) of the graph, the nM of the key illustrated is 1250 nM, 1000 nM, 750 nM, 500 nM, 250 nM, 300 nM, 150 nM, 125 nM, 75 nM, 62 nM, 31 nM, 15 nM, 7 nM, 3 nM, 1.9 nM, 0 nM; and (B) Slopes from linear regression at various inhibitor concentration. Individual curves were plotted as a fraction of the slope obtained in the absence of inhibitor. The arrow indicates the minimal concentration of inhibitor necessary for a complete inhibition of the ligation activity (150 nM).

FIG. 9 shows the conjugation of a tRNA methyltransferase, TrmJ using OaAEP1b-C247A-A351. SDS PAGE analysis of protein TrmJ-NAL conjugation with a short fluorescence peptide (GI-FITC). The reaction was carried out for an hour and sampled every 5, 10 and 30 minutes showing the time-dependent formation of the fluorescent-conjugate of TrmJ.

### DETAILED DESCRIPTION

The following detailed description refers to, by way of illustration, specific details and embodiments in which the invention may be practiced. These embodiments are described in sufficient detail to enable those skilled in the art to practice the invention. Other embodiments may be utilized and structural and logical changes may be made without departing from the scope of the invention.

Embodiments described below in context of the polypeptides, nucleic acids, vectors, host cells are analogously valid for the respective methods, and vice versa. The various embodiments are not necessarily mutually exclusive, as some embodiments can be combined with one or more other embodiments to form new embodiments.

Unless otherwise defined, all technical and scientific terms used herein have the same meaning as commonly understood by one of ordinary skill in the art. The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will prevail. “About”, as used herein in connection with numerical values refers to the referenced numerical value±10% or ±5%.

To date, constitutively active forms of either PALs or AEPs have only been able to be obtained via activation under acidic conditions [15-17]. This acidic-activation step leads to the introduction of a heterogeneous population of enzymes due to the multiple accessible activation sites present in the proenzyme, thus, limiting the quality and quantity of homogenous active PALs or AEPs obtained.

Accordingly, an object of the present invention is to provide methods capable of producing a protein ligase that is enzymatically active and does not require acid-activation, and more particularly to provide a constitutively active form of protein ligase that retains a level of catalytic activity similar to the acid-activated species.

Structural studies revealed that PALs and AEPs [18-22] share a similar overall fold formed by a core domain linked to a C-terminal cap domain via a flexible linker. The core domain consists of a six-stranded β-sheet surrounded by six α-helices located at its periphery, while the cap domain is formed by a suite of α helices [17,23,24]. An evolutionarily conserved glutamine residue at the N-terminus of the cap domain (Gln347 in OaAEP1b), inserts into the S1 pocket, keeping the pro-enzyme in an inactive state [17]. Upon activation at acidic pH values ranging from 4.0 to 4.5, the cap domain becomes separated from the core domain via electrostatic repulsion, facilitating cleavage in trans and exposing the enzyme active site to the solvent [19,25-27]. This cleavage allows binding by the PAL of polypeptide substrates containing the N/DX1X2 tripeptide motifs, where X1 is any residue besides Pro and X2 is a hydrophobic residue [26]. Such motifs are present at the N-terminus and within the linker region and cap domain of the proenzyme accounting for autoproteolysis activity observed at these sites. At acidic pH, hydrolysis is favoured, leading to the degradation of the cap domain and the N-terminus of the core domain. In vivo, acidic proteolytic activation occurs in the vacuole of cyclotide-producing plants and serves to regulate the activity of these enzymes endowed with proteolytic and cyclization activity [27-31].

In this regard, the present invention is based on the identification of molecular determinants governing the ability to express and purify stable constitutively active protein ligases that can be stored for months without significant activity loss. This advantageously removes the need for the low pH (acidic) proenzyme activation step which consequently eliminates the heterogeneity introduced by this procedure. Beneficially, the purification of stable constitutively active ligases in an expression system, constitutes a cost-effective way for the large-scale production of several hyperactive ligases. In turn, these stable constitutively active ligases will be convenient tools for various attractive industrial applications that require protein conjugation such as for the manufacturing of antibody-drug conjugates.

In particular, it was discovered that the molecular determinants governing the ability to express and purify stable constitutively active PALs are found in the linker and the first N-terminal helix of the cap domain (i.e. α6-helix region located at the N-terminal of the ligase cap domain), whereby retention of a portion of said first N-terminal helix of the cap domain enabled the purification of the protein from inclusion bodies without any severe precipitation and is important in maintaining protein stability in solution. In this regard, to produce an enzymatically active protein ligase (i.e. constitutively active protein ligase), without the need for any acid-activation step, the protein ligase can be recombinantly expressed with a truncated cap domain provided that a portion of the first N-terminal helix of the cap domain (e.g. α6-helix) is retained.

Accordingly, in various embodiments, there is provided a truncated polypeptide having protein ligase activity that is designed to be recombinantly expressed and purified in a constitutively active form for use in ligating or cyclizing at least two peptides.

The expression and purification of a constitutively active protein ligase as disclosed herein alleviates the need for a tedious activation step and any additional purification procedures. The expression and purification protocol leads to an enzyme endowed with comparable ligation kinetics to its acid-activated counterparts. Remarkably, compared to currently available acid-activation methods for protein ligase expression and purification, the yield of the constitutively active protein ligase disclosed herein is increased using the constructs and methods disclosed herein.

Accordingly, in various embodiments, the polypeptide having protein ligase activity as disclosed herein is a constitutively active protein ligase. In the context of the present disclosure, the term “constitutively active” refers to the polypeptide exhibiting enzymatic activity, more particularly protein ligase activity, without requiring proenzyme activation by cleavage. For example, a “constitutively active protein ligase” disclosed herein refers to a protein ligase that is enzymatically active independent of activation steps, such as acid activation, following expression and purification.

In various embodiments, the polypeptide having protein ligase activity as disclosed herein is a stable constitutively active protein ligase, whereby the polypeptide is stably expressed as a constitutively active protein ligase following a simple refolding step. In the context of the present disclosure, the term “stable” or “stability” refers to the polypeptide retaining protein ligase activity under storage conditions for a predetermined period of time (e.g. up to 2 years) without significant or detrimental loss of activity.

As illustrated in the working examples disclosed herein, the highly active PAL single mutant OaAEP1b-C247A (SEQ ID NO:2) was selected as a representative PAL for investigation in identifying the aforementioned molecular determinants due to the availability of a convenient bacterial recombinant expression system, while other hyperactive PALs require expression in insect cells systems [15-17]. In particular, constructs comprising the core domain, linker and cap domain without the signal peptide region of OaAEP1b-C247A (SEQ ID NO:3) was used for investigation. In this regard, the signal peptide region of OaAEP1b-C247A corresponds to amino acid residues positioned at 1-54; the core domain of OaAEP1b-C247A corresponds to amino acid residues positioned at 55-324; the linker of OaAEP1b-C247A corresponds to amino acid residues positioned at 325-347; and the cap domain of OaAEP1b-C247A corresponds to amino acid residues positioned at 348-474, wherein the numbering is in accordance with SEQ ID NO:1 (OaAEP1b). The α6-helix region within the cap domain of OaAEP1b-C247A corresponds to amino acid residues positioned at 350-361.

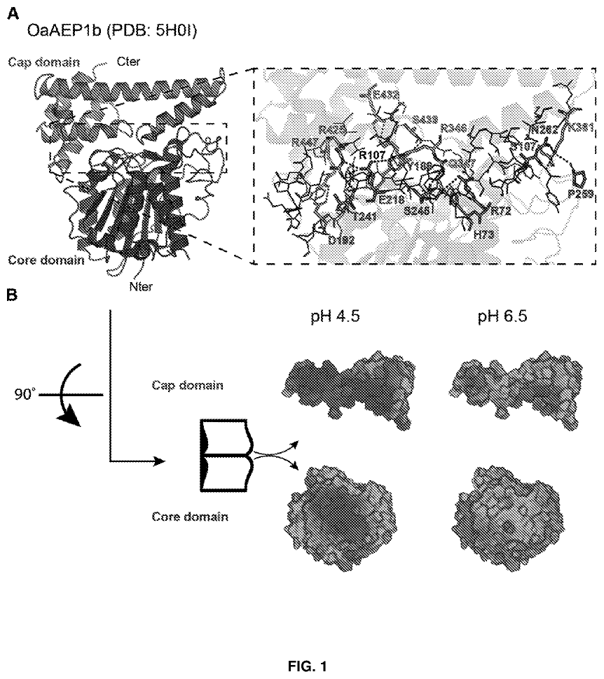

Several truncated constructs of the OaAEP1b-C247A proenzyme were designed and expressed retaining only portions of the linker (connecting the cap and core domain of the proenzyme) and the α6-helix region located at the N-terminal end of its cap domain (FIG. 1). Recombinant expression of the truncated constructs was carried out in E. coli, whereby all constructs were overexpressed in E. coli as inclusion bodies. Following a solubilization/refolding protocol, a truncated construct termed “OaAEP1b-C247A-Δ351” (SEQ ID NO: 4) could be overexpressed as inclusion bodies in an insoluble fraction, refolded and purified, and displayed a level of ligase activity comparable to the acid-activated OaAEP1b-C247A enzyme. In contrast, the other truncated constructs precipitated during the refolding procedure. The constitutively active truncated construct “OaAEP1b-C247A-Δ351” is able to be stored for up to two years at −80° C. and readily used for peptide cyclization and protein conjugation. Thus, this represents a cost-effective and faster way to produce large amounts of a hyperactive ligase in E. coli for various attractive biotechnological and industrial applications [1].

In the context of the present disclosure, the exemplified truncated construct termed “OaAEP1b-C247A-Δ351” relates to the amino acid sequence set forth in SEQ ID NO: 4, comprising the core domain, linker and cap domain with a C-terminal truncation denoted as 4351 referring to the deletion of amino acid residues at positions 352-474 such that amino acid residue D at position 351 forms the C-terminus of the amino acid sequence, wherein the position numbering is in accordance with SEQ ID NO:1.

Accordingly, the recombinant expression of a constitutively active protein ligase was confirmed by investigating systematic truncations along the α6 helix of OaAEP1b-C247A which penetrates into the enzyme active site. It was found that these truncations resulted in the protein ligase being expressed as inclusion bodies, demonstrating that the cap domain provides a set of polar interactions with the core domain that are important for soluble recombinant expression of the proenzyme. Moreover, constructs entirely devoid of the α6-helix displayed severe precipitation during the purification process. In contrast, construct OaAEP1b-C247A-Δ351, which retains a portion of α6-helix enabled the purification of the protein from inclusion bodies without any severe precipitation. This result supports the concept that the presence of a portion of the x6-helix is crucial in maintaining protein stability in solution.

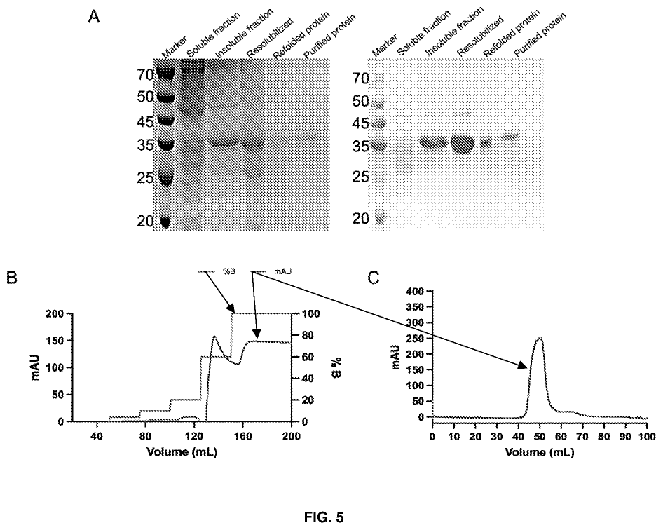

Despite having retained a portion of the cap domain, the truncated construct OaAEP1b-C247A-Δ351 was shown to retain high enzymatic activity in an intramolecular cyclization assay, with the complete conversion of the linear substrate to the cyclized product being detected (FIG. 6). Likewise, taking into account the amount of active enzymes, in an intermolecular ligation assay, the catalytic rate observed for the refolded OaAEP1b-C247A-Δ351 was shown to be comparable with its acid-activated counterpart. Nonetheless, the intermolecular ligation of two peptides and the conjugation assays were shown to be slower than intramolecular cyclization. An intramolecular cyclization reaction generally proceeds faster due to the incoming nucleophile being present in cis within the peptide substrate. In contrast, for an intermolecular ligation, a molar excess of electrophilic and nucleophilic substrate peptides is required for efficient catalysis of the reaction [10].

Further, the OaAEP1b-C247A-Δ351 construct was shown to provide an economical advantage compared to the original OaAEP1b-C247A construct that needs acid-activation to obtain an enzymatically competent form [16,17]. Specifically, using the OaAEP1b C247A proenzyme as the starting material [17], the final yield after acid-activation and purification is about 1-2 mg/L of culture, which is significantly lower than the obtained yield of refolded and active OaAEP1b-C247A-Δ351 enzyme of about 15 mg/L of culture. However, it is of note that scaling-up in the laboratory does not necessarily translate into an exact tenfold increase in yield, as large volumes of refolding buffers would have to be handled when using several litres of cell culture.

To summarise, there is provided a truncated protein ligase construct devoid of its inhibitory cap domain while retaining a portion of the α6-helix, that is expressed in a constitutively active form without acid-activation, and retains a level of catalytic activity acceptable, similar or better to the acid-activated species.

Further, there is also provided a method for producing a polypeptide having protein ligase activity, more particularly a constitutively active protein ligase, comprising culturing a host cell comprising a nucleic acid molecule encoding the polypeptide under conditions that allows expression of the polypeptide, and isolating said expressed polypeptide from the host cell or culture medium to obtain a polypeptide having protein ligase activity.

The terms “peptide”, “polypeptide”, and “protein” are used interchangeably to refer to polymers of amino acids of any length connected by peptide bonds. The polymer may comprise modified amino acids, it may be linear or branched, and it may be interrupted by non-amino acids. The terms also encompass an amino acid polymer that has been modified naturally or artificially; for example, by disulfide bond formation, glycosylation, lipidation, acetylation, phosphorylation, or any other manipulation, such as conjugation to a labeling moiety. However, in various embodiments, these terms relate to polymers of naturally occurring amino acids, as defined below, which may optionally be modified as defined above, but does not comprise non-amino acid moieties in the polymer backbone. The polypeptides, as disclosed herein, can have a length of at least 250 amino acids (aa), preferably at least 295 aa. In various embodiments, the polypeptides, as defined herein, can have a length of 295 to 450, 295 to 425, 295 to 400, 295 to 375, 295 to 350, or 295 to 320 aa.

The term “amino acid” refers to natural and/or unnatural or synthetic amino acids, including both the D and L optical isomers, amino acid analogs (for example norleucine is an analog of leucine) and derivatives known in the art. The term “naturally occurring amino acid”, as used herein, relates to the 20 naturally occurring L-amino acids, namely Gly, Ala, Val, Leu, Ile, Phe, Cys, Met, Pro, Thr, Ser, Glu, Gln, Asp, Asn, His, Lys, Arg, Tyr, and Trp. The term “peptide bond” refers to a covalent amide linkage formed by loss of a molecule of water between the carboxyl group of one amino acid and the amino group of a second amino acid. Generally, in all formulae depicted herein, the peptides are shown in the N- to C-terminal orientation. All amino acid residues are generally referred to herein by reference to their one letter code and, in some instances, their three-letter code. This nomenclature is well known to those skilled in the art and used herein as understood in the field.

The polypeptide disclosed herein and produced by the methods disclosed herein, exhibit protein ligation activity, i.e., it is capable of forming a peptide bond between two amino acid residues, with these two amino acid residues being located on the same or different peptides or proteins, preferably on the same peptide or protein so that said ligation activity cyclizes said peptide or protein. Accordingly, in various embodiments, the polypeptide as disclosed herein has cyclase activity. In various embodiments, this protein ligation or cyclase activity includes an endopeptidase activity, i.e. the polypeptide form a peptide bond between two amino acid residues following cleavage of an existing peptide bond. This means that cyclization need not to occur between the termini of a given peptide but can also occur between internal amino acid residues, with the amino acids C-terminal or N-terminal to the amino acid used for cyclization being cleaved off. In a preferred embodiment, the polypeptide forms a cyclized peptide by ligating the N-terminus to an internal amino acid and cleaving the remaining C-terminal amino acids. In particular, the polypeptide as disclosed herein is “Asx-specific” in that the amino acid C-terminal to which ligation occurs, i.e. the C-terminal end of the peptide that is ligated, is either asparagine (Asn or N) or aspartic acid (Asp or D), preferably asparagine. In various embodiments, a polypeptide as disclosed herein also has ligation activity for a peptide that has a C-terminal Asx (N or D) residue that is amidated, i.e. the C-terminal carboxy group is replaced by an amide group. This amide group is cleaved off in the course of the ligation reaction. Accordingly, such amidated peptide substrates, while still being ligated/cyclized, do not comprise the naturally occurring tripeptide motif NHV.

In various embodiments, the polypeptide can ligate a given peptide with an efficiency of 50% or more, 60% or more, 70% or more, 80% or more, preferably 90% or more. The protein ligation, preferably cyclization, reaction is preferably comparably fast, i.e. said polypeptide can cyclize a given peptide with a Kₘ of 500 μM or less, preferably 250 μM or less; and/or a K_{cat} of at least 0.05 s⁻¹, preferably at least 0.5 s⁻¹, more preferably at least 1.0 s⁻¹, most preferably at least 1.5 s⁻¹. In various embodiments, the polypeptides satisfy both requirements, i.e. the Kₘ and K_{cat} requirement. Methods to determine such Michaelis-Menten kinetics are well known in the art and can be routinely applied by those skilled in the art. In various embodiments, the polypeptides disclosed herein have at least 50%, more preferably at least 70, most preferably at least 90% of the protein ligase activity compared to its acid-activated counterpart.

In various embodiments, the polypeptide having protein ligase activity comprises or consists of an amino acid sequence as set forth in SEQ ID NO: 3 (OaAEP1b C247A: core domain+linker+cap domain) or variants thereof, comprising a C-terminal truncation (i.e. truncation within the cap domain) after amino acid position 351 (i.e. the start of the truncation may be at amino acid position 351 or any higher amino acid position within the cap domain), wherein position numbering is in accordance with SEQ ID NO:1 (OaAEP1b). In this regard, the amino acid residue at amino acid position 351 or any higher amino acid position defines the C-terminus of the amino acid sequence and polypeptide.

In various embodiments, the polypeptides disclosed herein include variants of the amino acid sequence as set forth in SEQ ID NO: 3 (OaAEP1b C247A: core domain+linker+cap domain) or variants thereof, comprising a C-terminal truncation (i.e. truncation within the cap domain) after amino acid position 351, wherein position numbering is in accordance with SEQ ID NO:1 (OaAEP1b).

The term “variants” refers to a polypeptide having protein ligase activity comprising a modification or alteration in addition to the defined C-terminal truncation. The modification or alteration may be a substitution, insertion, and/or deletion, at one or more (e.g., one or several) positions compared to the reference amino acid sequence other than those amino acid positions corresponding to the C-terminal truncation. A substitution means replacement of the amino acid occupying a position with a different amino acid; a deletion means removal of the amino acid occupying a position; and an insertion means adding an amino acid adjacent to and immediately following the amino acid occupying a position.

In this regard, variants of the amino acid sequence as set forth in SEQ ID NO: 3 herein may comprise a substitution, deletion, and/or insertion at one or more amino acid positions (excluding those positions corresponding to the C-terminal truncation) compared to the reference amino acid sequence. The amino acid changes may be of a minor nature, that is conservative amino acid substitutions or insertions that do not significantly affect the folding and/or activity of the protein. Such polypeptide variants are, for example, further developed by targeted genetic modification, i.e. by way of mutagenesis methods, and optimized for specific purposes or with regard to special properties (for example, with regard to their catalytic activity, stability, etc.). It is to be understood that the various polypeptides variants having at least one of the aforementioned deletions and/or mutations, even if their amino acid sequences are not explicitly described herein for the sake of conciseness, are contemplated to be within the scope of the present invention.

It will be appreciated that the “variants” disclosed herein share a % sequence identity or % sequence homology with the reference amino acid sequence set forth in SEQ ID NO: 3.

Accordingly, in various embodiments, the polypeptide comprises or consists of an amino acid sequence that is at least 55%, 60%, 65%, 70%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.25%, or 99.5% identical or homologous to the amino acid sequence set forth in SEQ ID NO:3 over its entire length, wherein the amino acid sequence comprises a C-terminal truncation after amino acid position 351, wherein position numbering is in accordance with SEQ ID NO:1.

In various embodiments, the polypeptide comprises or consists of an amino acid sequence that shares at least 55, preferably at least 60, preferably at least 70, more preferably at least 80, most preferably at least 90% sequence identity with the amino acid sequence set forth in SEQ ID NO:3 over its entire length, or the polypeptide comprises or consists of an amino acid sequence that shares at least 70, preferably at least 80, preferably at least 90, more preferably at least 95% sequence homology with the amino acid sequence set forth in SEQ ID NO: 3 over its entire length, wherein the amino acid sequence comprises a C-terminal truncation after amino acid position 351, wherein position numbering is in accordance with SEQ ID NO:1.

The identity of amino acid sequences is generally determined by means of a sequence comparison. This sequence comparison is based on the BLAST algorithm that is established in the existing art and commonly used (cf. for example Altschul et al. (1990) “Basic local alignment search tool”, J. Mol. Biol. 215:403-410, and Altschul et al. (1997): “Gapped BLAST and PSI-BLAST: a new generation of protein database search programs”; Nucleic Acids Res., 25, p. 3389-3402) and is effected in principle by mutually associating similar successions of nucleotides or amino acids in the nucleic acid sequences and amino acid sequences, respectively. A tabular association of the relevant positions is referred to as an “alignment.” Sequence comparisons (alignments), in particular multiple sequence comparisons, are commonly prepared using computer programs which are available and known to those skilled in the art.

A comparison of this kind also allows a statement as to the similarity to one another of the sequences that are being compared. This is usually indicated as a percentage identity, i.e. the proportion of identical nucleotides or amino acid residues at the same positions or at positions corresponding to one another in an alignment. The more broadly construed term “homology”, in the context of amino acid sequences, also incorporates consideration of the conserved amino acid exchanges, i.e. amino acids having a similar chemical activity, since these usually perform similar chemical activities within the protein. The similarity of the compared sequences can therefore also be indicated as a “percentage homology” or “percentage similarity.” Indications of identity and/or homology can be encountered over entire polypeptides, or only over individual regions. Homologous and identical regions of various nucleic acid sequences or amino acid sequences are therefore defined by way of matches in the sequences. Such regions often exhibit identical functions. They can be small, and can encompass only a few nucleotides or amino acids. Small regions of this kind often perform functions that are essential to the overall activity of the protein. It may therefore be useful to refer sequence matches only to individual, and optionally small, regions. Unless otherwise indicated, however, indications of identity and homology herein refer to the full length of the respectively indicated nucleic acid sequence or amino acid sequence.

Accordingly, the “variants” disclosed herein share a % sequence identity or % sequence homology with the reference amino acid sequence set forth in SEQ ID NO: 3, such that the variants may include or be derived from PALs or AEPs other than OaAEP1b-C247A. In various embodiments, the variants may include or be derived from the known PALs of VyPAL2 (SEQ ID NO:7 or 8), butelase-1 (SEQ ID NO:9 or 10), butelase-2 (SEQ ID NO: 11 or 12), VcPAL (SEQ ID NO: 13 or 14), or VuPAL (SEQ ID NO: 15 or 16).

In consideration of the structural similarities shared between PALs and AEPs mentioned above, it will be appreciated that the identified molecular determinants and results illustrated using the exemplified protein ligase construct of OaAEP1b-C247A can be extended to other PALs and AEPs, whereby truncated constructs of other PALs or AEPs that retain a portion of said first N-terminal helix of the cap domain can be suitably designed and expressed as constitutively active ligases that are shown to retain ligase activity at an acceptable or comparable level with its acid-activated counterpart. In particular, while the findings described herein were derived in the context of the amino acid sequence of OaAEP1b-C247A (SEQ ID NO: 3), one skilled in the art would readily appreciate that the findings are applicable to other PALS or AEPs, which share a sequence identity or homology with OaAEP1b-C247A.

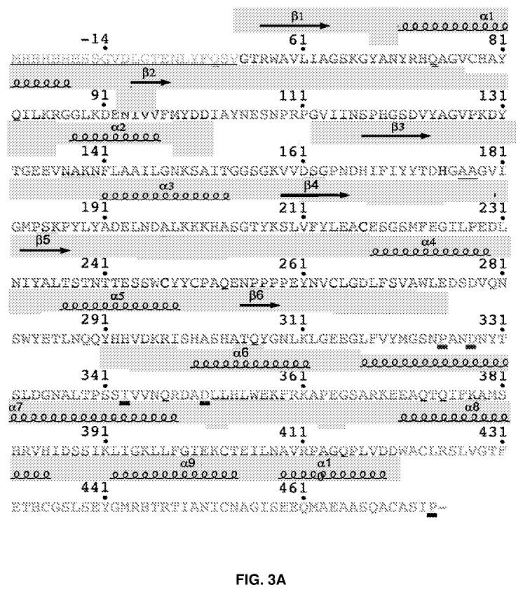

For example, FIG. 3B is a sequence alignment of VyPAL2, OaAEP1b, butelase-1 and VuPAL showing the conservation of the region relating to the linker and N-terminal of the cap domain (i.e. amino acids at positions 325-361 in accordance with the numbering of SEQ ID NO:1), where the truncation of the polypeptide may be after the conserved residue D351, such as within the first N-terminal helix of the cap domain (i.e. α6-helix). As such, a person skilled in the art would reasonably and plausibly expect that the experimental results shown with regard to OaAEP1b-C247A are applicable in obtaining truncated constructs of VyPAL2, VuPAL and butelase-1 that are constitutively active.

Moreover, it will be appreciated that determinants for the protein ligase activity of PALs and AEPs are known to those skilled in the art and described, for example, in WO 2020/226572 A1, the contents of which is incorporated herein in its entirety, such that those skilled in the art would understand which amino acid residues and motifs are crucial for maintaining activity within the core domain. In WO 2020/226572 A1, the molecular determinants governing asparaginyl endopeptidases and ligases activity were primarily found, based upon analysis and investigation of the protein ligase “VyPAL2”. In this regard, the molecular determinants were found in the amino acid composition of the substrate-binding grooves flanking the S1 pocket, in particular the LAD1 and LAD2 (ligase activity determinants 1 and 2) that are centered around the S2 and S1′ pockets, respectively. For an efficient peptide asparaginyl ligase, the first position of LAD1 is preferably bulky and aromatic, such as W/Y, and the second position hydrophobic, such as V/I/C/A but not G. For LAD2, it was found that GA/AA/AP dipeptides are favored. A bulky residue such as Y is disadvantageous at the first position of LAD2, as it is likely to destabilize the acyl-enzyme intermediate, by affecting the binding affinity of substrates and controlling the accessibility of water molecules and by increasing the dissociation rate of the cleaved peptide tail after the N/D residue. As shown in FIG. 3A, OaAEP1b-C247A comprises amino acid residues WCY for LAD1 at positions 246-248, and amino acid residues AA for LAD2 at positions 177 and 178.

In various embodiments, the polypeptide having protein ligase activity comprises or consists of an amino acid sequence as set forth in SEQ ID NO: 8 (VyPAL2) comprising a C-terminal truncation (i.e. truncation within the cap domain) after amino acid position 351, wherein position numbering is in accordance with SEQ ID NO:1 (OaAEP1b).

In various embodiments, the polypeptide having protein ligase activity comprises or consists of an amino acid sequence as set forth in SEQ ID NO: 10 (butelase-1) comprising a C-terminal truncation (i.e. truncation within the cap domain) after amino acid residue 351, wherein position numbering is in accordance with SEQ ID NO:1 (OaAEP1b).

In various embodiments, the polypeptide having protein ligase activity comprises or consists of an amino acid sequence as set forth in SEQ ID NO: 12 (butelase-2) comprising a C-terminal truncation (i.e. truncation within the cap domain) after amino acid residue 351, wherein position numbering is in accordance with SEQ ID NO:1 (OaAEP1b).

In various embodiments, the polypeptide having protein ligase activity comprises or consists of an amino acid sequence as set forth in SEQ ID NO: 14 (VcPAL) comprising a C-terminal truncation (i.e. truncation within the cap domain) after amino acid residue 351, wherein position numbering is in accordance with SEQ ID NO:1 (OaAEP1b).

In various embodiments, the polypeptide having protein ligase activity comprises or consists of an amino acid sequence as set forth in SEQ ID NO: 16 (VuPAL) comprising a C-terminal truncation (i.e. truncation within the cap domain) after amino acid residue 351, wherein position numbering is in accordance with SEQ ID NO:1 (OaAEP1b).

Accordingly, there is provided a polypeptide having protein ligase activity, more preferably a constitutively active protein ligase, comprising or consisting of:
(i) an amino acid sequence as set forth in SEQ ID NO: 3 (OaAEP1b C247A: core domain+linker+cap domain);
(ii) an amino acid sequence that shares at least 55, preferably at least 60, preferably at least 70, more preferably at least 80, most preferably at least 90% sequence identity with the amino acid sequence set forth in (i) over its entire length; or
(iii) an amino acid sequence that shares at least 70, preferably at least 80, more preferably at least 90, most preferably at least 95% sequence homology with the amino acid sequence set forth in (i) over its entire length,
   wherein the amino acid sequence of (i)-(iii) comprise a C-terminal truncation after amino acid position 351, and wherein position numbering is in accordance with SEQ ID NO:1 (OaAEP1b).

In various embodiments, the polypeptide is a recombinant polypeptide comprising or consisting of the amino acid sequence (i), (ii) or (iii), and is in an enzymatically active isoform, whereby the polypeptide is a recombinantly expressed polypeptide.

In various embodiments, the polypeptide having protein ligase activity is a non-naturally occurring polypeptide (i.e. it is one not found in nature).

In various embodiments, the polypeptide disclosed herein is an isolated polypeptide, that is a polypeptide in isolated form, more specifically, is directed to an isolated polypeptide comprising or consisting of the amino acid sequence (i), (ii) or (iii). The term “isolated” as used herein, relates to the polypeptide in a form where it has been at least partially separated or removed from other cellular components it may associate with.

The term “truncation” as used herein refers to a removal of one or more amino acid residues from the amino acid sequence of the reference polypeptide (e.g. SEQ ID NO:3 or variants thereof). In this regard, a C-terminal truncation refers to the removal of one or more amino acid residues from the C-terminal end (i.e. cap domain) of the amino acid sequence of the reference polypeptide, provided that a portion of the cap domain is retained. In various embodiments, the portion of the cap domain comprises or consists of amino acid residues at the positions corresponding to positions 348-351 of SEQ ID NO:1.

The phrase “C-terminal truncation after amino acid position 351” refers to the truncation of the C-terminal segment of the reference amino acid sequence (e.g. C-terminal cap domain of SEQ ID NO:3) retaining the amino acid residue designated at position 351, and the truncation starting at a higher amino acid residue closer to and moving in the direction of the C-terminus of the amino acid sequence relative to position 351, wherein position numbering is in accordance with SEQ ID NO:1 (OaAEP1b). In other words, the amino acid sequence (i)-(iii) is truncated at any amino acid position after the amino acid residue at position 351, such as the amino acid residue at position 352, 353, 354, 355, 356, 357, 358, 359, 360, 361 etc. up to the penultimate amino acid residue of the cap domain. Accordingly, the amino acid position that defines the start of the truncation refers to the amino acid residue that forms the C-terminus of the amino acid sequence and polypeptide, for example, if the truncation starts at amino acid position 351, then the amino acid residue designated at position 351 is the C-terminus amino acid of the polypeptide with a free carboxyl group, with the subsequent amino acid residues of the cap domain, being deleted, wherein position numbering is in accordance with SEQ ID NO:1 (OaAEP1b).

The term “C-terminus” as used herein refers to the terminal amino acid residue of a polypeptide having a free carboxyl group, where the carboxyl group in non-C-terminus amino acid residues normally forms part of the covalent backbone of the polypeptide. The term “N-terminus” as used herein refers to the terminal amino acid residue of a polypeptide having a free amine group, where the amine group in non-N-terminus amino acid residues normally forms part of the covalent backbone of the polypeptide. “N-terminal” refers to the region of a polypeptide or domain that is adjacent to the N-terminus of the polypeptide or domain, and “C-terminal” refers to the region of a polypeptide or domain that is adjacent to the C-terminus of the polypeptide or domain.

In various embodiments, the C-terminal truncation starts at an amino acid residue positioned within the first N-terminal helix of the cap domain, more particularly the α6-helix, provided that a portion of the first N-terminal helix of the cap domain is retained. In various embodiments, the portion of the first N-terminal helix comprises or consists the two amino acid residues AD at the positions corresponding to positions 350 and 351 of SEQ ID NO:1.

Alternative truncations starting within the cap domain, or first N-terminal helix, are also contemplated to produce a constitutively active peptide ligase. In various embodiments, the C-terminal truncation starts at an amino acid residue at a position between 351 to 361, inclusive, wherein position numbering is in accordance with SEQ ID NO:1 (OaAEP1b).

In various embodiments, the amino acid sequence of (i)-(iii) comprise a C-terminal truncation at amino acid position 351, wherein position numbering is in accordance with SEQ ID NO:1 (OaAEP1b), and wherein the amino acid residue at position 351 is the C-terminus of the polypeptide.

FIG. 3B shows sequence and structural conservation within the linker and cap domain, especially the first N-terminal helix of the cap domain, particularly the amino acid residues at positions 325-351, more particularly the amino acid residues at positions 344-351 of SEQ ID NO: 1, between OaAEP1b, VyPAL2, butelase-1, and VuPAL.

In various embodiments, the polypeptide comprises the amino acid residue Q at the position corresponding to position 347 of SEQ ID NO:1; and/or the polypeptide comprises the amino acid residue R or H at the position corresponding to position 348 of SEQ ID NO:1; and/or the amino acid residue D at the position corresponding to position 349 and 351 of SEQ ID NO:1, and/or the amino acid residue A at the position corresponding to position 350 of SEQ ID NO:1. In various embodiments, the polypeptide comprise at least two, preferably at least three, more preferably all four of the above indicated residues at the given or corresponding positions.

In various embodiments, the polypeptide comprises the amino acid residue D at the position corresponding to position 349 and 351 of SEQ ID NO:1, and the amino acid residue A at the position corresponding to position 350 of SEQ ID NO:1.

In various embodiments, the polypeptide comprises the amino acid residue A or V at the position corresponding to position 344 of SEQ ID NO:1; and/or the amino acid residue V or I at the position corresponding to position 345 of SEQ ID NO:1; and/or the amino acid residue V, N, H or S at the position corresponding to position 346 of SEQ ID NO:1. In various embodiments, the polypeptide comprise at least two, more preferably all three of the above indicated residues at the given or corresponding positions.

In various embodiments, the polypeptide comprises the amino acid residue P at the position corresponding to position 325 of SEQ ID NO:1; and/or the amino acid residue A at the position corresponding to position 326 of SEQ ID NO:1; and/or the amino acid residue N at the position corresponding to positions 327 and 329 of SEQ ID NO:1; and/or the amino acid residue D at the position corresponding to position 328 of SEQ ID NO:1; and/or the amino acid residue N at the position corresponding to position 336 of SEQ ID NO:1. In various embodiments, the polypeptide comprise at least one, more preferably at least 3, 4, 5 or all 6 of the above indicated residues at the given or corresponding positions.

In various embodiments, the polypeptide comprises or consists of an amino acid sequence as set forth in SEQ ID NO: 4 (OaAEP1b-C247A-Δ351) or variants thereof, wherein the amino acid at position 351 is the C-terminus of the polypeptide.

In various embodiments, said polypeptide may comprise a tag to facilitate isolation and purification of the polypeptide, without interfering with the folding and the function of the polypeptide. In various embodiments, the polypeptide further comprises an affinity tag at the N-terminal of the amino acid sequence as set forth in (i)-(iii), more particularly the affinity tag is positioned at or proximate to the N-terminus of the amino acid sequence as set forth in (i)-(iii). In various embodiments, the affinity tag includes, but is not limited to, an AviTag, His-tag or Strep-tag. In various embodiments, the affinity tag is a His-tag. In various embodiments, the His-tag is a hexahistidine tag.

In various embodiments, a cleavage sequence is included at the N-terminal of the amino acid sequence as set forth in (i)-(iii) that is cleaved by a site-specific protease, more particularly the cleavage sequence is positioned at or proximate to the N-terminus of the amino acid sequence as set forth in (i)-(iii). In various embodiments, the cleavage sequence includes, but is not limited to, a thrombin cleavage sequence, an enterokinase cleavage sequence, a PreScission cleavage sequence, a 3C cleavage sequence, a factor Xa cleavage sequence, or a TEV cleavage sequence. In various embodiments, the cleavage sequence is a TEV cleavage sequence.

In various embodiments, the polypeptide comprises an affinity tag and a cleavage sequence positioned at the N-terminal of the amino acid sequence as set forth in (i)-(iii), more particularly the affinity tag and the cleavage sequence are positioned at the N-terminus of the amino acid sequence as set forth in (i)-(iii). In various embodiments, the cleavage sequence is positioned between the affinity tag and the amino acid sequence as set forth in (i)-(iii). In various embodiments, the polypeptide comprises a His-tag and a TEV cleavage site comprising or consisting of an amino acid sequence as set forth in SEQ ID NO: 5 or variants thereof, positioned at the N-terminus of amino acid sequence as set forth in (i)-(iii).

In various embodiments, said polypeptide comprises or consists of an amino acid sequence as set forth in SEQ ID NO: 6 (His-tag+TEV cleavage site+OaAEP1b-C247A-Δ351) or variants thereof, wherein the amino acid residue at position 351 is the C-terminus of the polypeptide.

All embodiments disclosed herein in relation to the polypeptides are applicable to the methods disclosed herein and vice versa.

In the methods disclosed herein, the step of culturing may comprise recombinantly expressing the polypeptide disclosed herein, which refers to the expression of said polypeptide by recombinant DNA technology, wherein the polypeptide may be a recombinant polypeptide, i.e. polypeptide produced in a genetically engineered organism that does not naturally produce said polypeptide.

Accordingly, there is provided nucleic acid molecules encoding the polypeptides disclosed herein.

The term “nucleic acid molecule” or “nucleic acid” as used herein refers to a polymeric form of nucleotides of any length, either deoxyribonucleotides or ribonucleotides, or analogs thereof. Nucleic acid molecules may have any three-dimensional structure, and may perform any function, known or unknown. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A nucleic acid molecule may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labelling component.

The nucleic acid molecules can be DNA molecules or RNA molecules. They can exist as an individual strand, as an individual strand complementary to said individual strand, or as a double strand. With DNA molecules in particular, the sequences of both complementary strands in all three possible reading frames are to be considered in each case. Also to be considered is the fact that different codons, i.e. base triplets, can code for the same amino acids, so that a specific amino acid sequence can be coded by multiple different nucleic acids. As a result of this degeneracy of the genetic code, all nucleic acid sequences that can encode one of the above-described polypeptides are included in this subject of the invention. The skilled artisan is capable of unequivocally determining these nucleic acid sequences, since despite the degeneracy of the genetic code, defined amino acids are to be associated with individual codons. The skilled artisan can therefore, proceeding from an amino acid sequence, readily ascertain nucleic acids coding for that amino acid sequence. In addition, in the context of nucleic acids molecules disclosed herein one or more codons can be replaced by synonymous codons. This aspect refers in particular to heterologous expression of the enzymes contemplated herein. For example, every organism, e.g. a host cell of a production strain, possesses a specific codon usage. “Codon usage” is understood as the translation of the genetic code into amino acids by the respective organism. Bottlenecks in protein biosynthesis can occur if the codons located on the nucleic acid are confronted, in the organism, with a comparatively small number of loaded tRNA molecules. Also, it codes for the same amino acid, the result is that a codon becomes translated in the organism less efficiently than a synonymous codon that codes for the same amino acid. Because of the presence of a larger number of tRNA molecules for the synonymous codon, the latter can be translated more efficiently in the organism.

By way of methods commonly known today such as, for example, chemical synthesis or the polymerase chain reaction (PCR) in combination with standard methods of molecular biology or protein chemistry, a skilled artisan has the ability to manufacture, on the basis of known DNA sequences and/or amino acid sequences, the corresponding nucleic acids all the way to complete genes. Such methods are known, for example, from Sambrook, J., Fritsch, E. F., and Maniatis, T, 2001, Molecular cloning: a laboratory manual, 3rd edition, Cold Spring Laboratory Press.

In various embodiments, the nucleic acid molecule encoding the polypeptide disclosed herein is comprised within a vector.

Accordingly, there is provided a vector comprising a nucleic acid molecule encoding the polypeptide as disclosed herein. The vector may further comprise regulatory elements for controlling expression of said nucleic acid molecule.

As used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors can direct the expression of genes to which they are operatively-linked. Such vectors are referred to herein as “expression vectors.” Common expression vectors of utility in recombinant DNA techniques are often in the form of plasmids.

In various embodiments, the vector comprising the nucleic acid molecule encoding the polypeptide as disclosed herein is an expression vector.

In this regard, vectors enable said nucleic acid to be established as a stable genetic element in a species or a cell line over multiple generations or cell divisions. In particular when used in bacteria, vectors are special plasmids, i.e. circular genetic elements. In the context herein, a nucleic acid disclosed herein is cloned into a vector. Included among the vectors are, for example, those whose origins are bacterial plasmids, viruses, or bacteriophages, or predominantly synthetic vectors or plasmids having elements of widely differing derivations. Using the further genetic elements present in each case, vectors are capable of establishing themselves as stable units in the relevant host cells over multiple generations. They can be present extra chromosomally as separate units, or can be integrated into a chromosome resp. into chromosomal DNA.

Recombinant expression vectors can comprise a nucleic acid molecule disclosed herein in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety.

Expression vectors encompass nucleic acid sequences which are capable of replicating in the host cells, by preference microorganisms, particularly preferably bacteria, that contain them, and expressing therein a contained nucleic acid. In various embodiments, the vectors disclosed herein thus also contain regulatory elements that control expression of the nucleic acids encoding a polypeptide disclosed herein. Expression is influenced in particular by the promoter or promoters that regulate transcription. Expression can occur in principle by means of the natural promoter originally located in front of the nucleic acid to be expressed, but also by means of a host-cell promoter furnished on the expression vector or also by means of a modified, or entirely different, promoter of another organism or of another host cell. In the present case at least one promoter for expression of a nucleic acid as contemplated herein is made available and used for expression thereof. Expression vectors can furthermore be regulated, for example by way of a change in culture conditions or when the host cells containing them reach a specific cell density, or by the addition of specific substances, in particular activators of gene expression. One example of such a substance is the galactose derivative isopropyl-beta-D-thiogalactopyranoside (IPTG), which is used as an activator of the bacterial lactose operon (lac operon).

Accordingly, there is also provided a host cell, preferably a non-human host cell, containing the nucleic acid encoding the polypeptide or a vector containing the nucleic acid encoding the polypeptide for use in the methods disclosed herein to recombinantly express said polypeptide disclosed herein. In particular, the nucleic acid molecule or vector containing said nucleic acid molecule may be transformed or transfected into an organism, which then represents the host cell. Methods for the transformation or transfection of cells are established in the existing art and are sufficiently known to the skilled artisan.

In this regard, the nucleic acid molecule disclosed herein may be comprised in an “expression construct” which refers to a functional unit built in the vector for the purpose of recombinantly expressing the polypeptides disclosed herein, when introduced into an appropriate host cell.

All cells are in principle suitable as host cells, i.e. prokaryotic or eukaryotic cells. Those host cells that can be manipulated in genetically advantageous fashion, e.g. as regards transformation using the nucleic acid or vector and stable establishment thereof, are preferred, for example single-celled fungi or bacteria. In addition, preferred host cells are notable for being readily manipulated in microbiological and biotechnological terms. This refers, for example, to easy culturability, high growth rates, low demands in terms of fermentation media, and good production and secretion rates for foreign proteins. The polypeptides can furthermore be modified, after their manufacture, by the cells producing them, for example by the addition of sugar molecules, formylation, amination, etc. Post-translation modifications of this kind can functionally influence the polypeptide.

Further embodiments are represented by those host cells whose activity can be regulated on the basis of genetic regulation elements that are made available, for example, on the vector, but can also be present a priori in those cells. They can be stimulated to expression, for example, by controlled addition of chemical compounds that serve as activators, by modifying the culture conditions, or when a specific cell density is reached. This makes possible economical production of the proteins contemplated herein. One example of such a compound is IPTG.

In various embodiments, the host cell is a prokaryotic or bacterial cell, such as an E. coli cell. Bacteria are notable for short generation times and few demands in terms of culturing conditions. As a result, economical culturing methods resp. manufacturing methods can be established. In addition, the skilled artisan has ample experience in the context of bacteria in fermentation technology. Gram-negative or Gram-positive bacteria may be suitable for a specific production instance, for a wide variety of reasons to be ascertained experimentally in the individual case, such as nutrient sources, product formation rate, time requirement, etc.

The host cells disclosed herein may be modified in terms of their requirements for culture conditions, can comprise other or additional selection markers, or can also express other or additional proteins. They can, in particular, be those host cells that transgenically express multiple proteins or enzymes.

In various embodiments, the host cell is a eukaryotic cell, which is characterized in that it possesses a cell nucleus. In contrast to prokaryotic cells, eukaryotic cells are capable of post-translationally modifying the protein that is formed. Examples thereof are fungi such as Actinomycetes, or yeasts such as Saccharomyces or Kluyveromyces or insect cells, such as Sf9 cells. This may be particularly advantageous, for example, when the proteins, in connection with their synthesis, are intended to experience specific modifications made possible by such systems. Among the modifications that eukaryotic systems carry out in particular in conjunction with protein synthesis are, for example, the bonding of low-molecular-weight compounds such as membrane anchors or oligosaccharides. In various embodiments, the host cells are thus eukaryotic cells, such as insect cells, for example Sf9 cells.

In various embodiments, the eukaryotic host cell is a mammalian cell. The mammalian cell can include, but are not limited to a human, simian, murine, mice, rat, monkey, rabbit, rodent, hamster, goat, bovine, sheep or pig cells. In various embodiments, the eukaryotic host cell is a cell from a cell line including, but are not limited to Chinese hamster ovary (CHO) cells, murine myeloma cells such as NSO and Sp2/0 cells, COS cells, Hela cells and human embryonic kidney (HEK-293) cells.

In various embodiments, the eukaryotic host cell is a human embryonic kidney (HEK-293) cell, more preferably a human Expi293 cell.

In various embodiments, the eukaryotic host cell is a CHO cell, preferably a ExpiCHO cell.

The host cells disclosed herein are cultured in a usual manner, for example in discontinuous or continuous systems. In the former case a suitable nutrient medium is inoculated with the host cells, and the product is harvested from the medium after a period of time to be ascertained experimentally. Continuous fermentations are notable for the achievement of a flow equilibrium in which, over a comparatively long period of time, cells die off in part but are also in part renewed, and the protein formed can simultaneously be removed from the medium.

Accordingly, the methods disclosed herein comprise the step of culturing a host cell comprising a nucleic acid molecule encoding the polypeptide under conditions that allows expression of the polypeptide, wherein the polypeptide comprises of:
(i) an amino acid sequence as set forth in SEQ ID NO: 3 (OaAEP1b C247A core domain+linker+cap domain);
(ii) an amino acid sequence that shares at least 55, preferably at least 60, preferably at least 70, more preferably at least 80, most preferably at least 90% sequence identity with the amino acid sequence of (i) over its entire length; or
(iii) an amino acid sequence that shares at least 70, preferably at least 80, more preferably at least 90, most preferably at least 95% sequence homology with the amino acid sequence of (i) over its entire length,

wherein the amino acid sequence of (i)-(iii) comprise a C-terminal truncation after amino acid position 351, wherein position numbering is in accordance with SEQ ID NO:1 (OaAEP1b).

In various embodiments, the host cell comprises a nucleic acid encoding the polypeptide or a vector comprising said nucleic acid encoding the polypeptide.

In various embodiments, the host cell is an E. coli cell.

In various embodiments, the host cell (e.g. E. coli) is cultured in a suitable culture medium (e.g. LB media). Culture conditions and mediums can be selected by those skilled in the art based on the host organism used by resorting to general knowledge and techniques known in the art. In various embodiments, the host cell is an E. coli cell and the culture conditions include culturing the E. coli cells in a culture medium, preferably LB media, at a temperature of about 37° C. until a desired optical density (OD) is reached. In various embodiments, the expression of the polypeptide can be induced by IPTG, however it will be appreciated that other known expression induction methods may be used. The cultured host cells may be stored in the form of cell pellets at suitable conditions before further use or processing, for example the cell pellet may be stored at −80° C. until use. The term “cell pellets” as used herein indicates samples that contain cellular material that has been separated using centrifugation.

In various embodiments, the polypeptide disclosed herein may be isolated in various forms following the culturing step of the methods disclosed herein. Accordingly, the methods disclosed herein comprise the step of isolating the expressed polypeptide from the host cell or culture medium the host cell is cultured in. The term “isolated” as used herein, relates to the polypeptide in a form where it has been at least partially separated or removed from other cellular components it may associate with.

In various embodiments, the polypeptide may be isolated from the host cell or cell pellets through a variety of methods, including but not limited to cell lysis and centrifugation or other techniques that may involve density gradients or multiple steps of fractionation. For example, the host cells may be subjected to cell lysis using a suitable lysis buffer known in the art and centrifuged to obtain a cell pellet containing the polypeptide.

Accordingly, in various embodiments, the culturing step may be followed by lysing the host cells and isolating the expressed polypeptide from the lysed cells.

In various embodiments, the polypeptide is expressed as inclusion bodies in the host cell. In various embodiments, the host cell is an E. coli cell, and the polypeptide is expressed as bacterial inclusion bodies. As used herein the term “inclusion bodies” may refer to insoluble aggregates containing the expressed polypeptides present in the host cells.

Host cells containing the polypeptide expressed as inclusion bodies may be disrupted in a suitable buffer to obtain and extract the inclusion bodies as an insoluble fraction, for example, the host cell may be subjected to cell lysis using a suitable lysis buffer known in the art and then the insoluble fraction of the polypeptide separated and isolated from soluble material using centrifugation. In various embodiments, the lysis buffer has a pH 5-9, preferably pH 6-8 with a strength between 0.01-2.0 M. Salts like NaCl or KCl may also be included in the lysis buffer. In various embodiments, the lysis buffer comprises 100 mM Bis-Tris, 500 mM NaCl, 10% (v/v) glycerol and has a pH 6.5.

Accordingly, in various embodiments, the polypeptide is expressed as inclusion bodies, and the culturing step may be followed by the step of lysing the host cells and subsequently the step of isolating the expressed polypeptide from the lysed cells.

In various embodiments, the isolated polypeptides expressed as inclusion bodies can be denatured and subsequently refolded. These steps may ensure that the polypeptide disclosed herein is obtained with protein ligase activity. In particular, the isolated polypeptide may be denatured and solubilised by addition of a denaturant, such as urea. As used herein, the term “denaturant” refers to a compound that, in a suitable concentration in solution, is capable of changing the spatial configuration or conformation of polypeptides through alterations at the surface thereof so as to render the polypeptide soluble in the medium.

Accordingly, in various embodiments, the isolated polypeptide expressed as inclusion bodies may be solubilized and denatured using a suitable solubilization buffer containing a denaturant, such as urea. The conditions which process the said inclusion body with the said solubilization buffer are not specifically limited, so long as conditions (i.e. treatment temperature, the treatment time, and the like) are appropriately set so that the isolated polypeptide is solubilized and denatured by the solubilizing buffer according to the composition and pH of the solubilizing buffer. In various embodiments, the pH of the solubilizing buffer is 5-8, preferably about 6.5. In various embodiments, the solubilizing buffer comprises 50 mM Bis-Tris, 150 mM NaCl, 1 mM EDTA, 50 mM Glycine, 8 M urea at a pH 6.5. In various embodiments, the solubilization may be carried out at any temperature at which the polypeptide can be solubilized, preferably 2° C. to 40° C., more preferably 4° C. to 37° C., and further preferably about 4° C.

After the step of solubilization, the solubilized polypeptide may be re-folded into a polypeptide having protein ligase activity. The refolding step is not particularly limited, and conventionally known methods can be used. In various embodiments, the refolding step is performed by a series of dilution and dialysis method steps, well-known to a person skilled in the art. One or more refolding buffer solutions may be used for refolding and are not particularly limited, but promote or assist formation of a three-dimensional structure, that is, formation of an intermolecular or intramolecular disulfide bond. The term “refolding buffer” refers to compounds or a combination of compounds and/or conditions which assist during the process of correctly folding of a protein that is improperly folded, unfolded or denatured. Further, the refolding buffer helps in maintaining the pH of the solution during the process of refolding.

The refolding buffer solutions may contain an S—S bond formation promoting, for example, reduced glutathione (hereinafter also referred to as “GSH”), oxidized glutathione (hereinafter also referred to as “GSSG”), dithiothreitol (Hereinafter also referred to as “DTT”) or the like can be used. These may be used alone or in combination. The concentration of the SS bond formation promotion in the refolding buffer solution is not particularly limited, and may be set according to the type of the SS bond formation promotion used. For example, when GSH and GSSG are used as the SS bond formation promoting/adjuvant, the GSH concentration is preferably 1 mM to 5 mM, and the GSSG concentration is preferably 0.1 mM to 0.5 mM. In various embodiments, the pH of the refolding buffer solution is 5-8, preferably about 6.5. Furthermore, the refolding buffer may contain 50 mM to 500 mM salt, preferably about 100 to 150 mM. Examples of the salt include, but are not limited to NaCl, KCl, CaCl₂), and MgCl₂. In various embodiments, the refolding may be carried out at any temperature at which the polypeptide can be refolded into a polypeptide having protein ligase activity, preferably 2° C. to 40° C., more preferably 4° C. to 37° C., and further preferably about 4° C.

In various embodiments, the solubilized polypeptide is first diluted with a first refolding buffer, which may also be termed as a dilutant buffer, (e.g. containing 50 mM Bis-Tris, pH 6.5, 150 mM NaCl, 500 mM L-Arginine, 1 M urea, 0.25 mM L-Glutathione oxidized, 2.5 mM L-Glutathione reduced) to reduce the denaturant and protein concentration. The solubilized polypeptide may be diluted to about 10-100 fold or about 10-50 fold or about 10-25 fold with the first refolding buffer. The final protein concentration after dilution may be about 0.01-4 mg/mL, preferably about 2 mg/ml. The diluted polypeptide may then be dialysed with a second refolding buffer, which may also be termed as a dialyzing buffer (e.g. containing 50 mM Bis-Tris, pH 6.5, 150 mM NaCl, 200 mM L-Arginine, 0.125 mM L-Glutathione oxidized, 1.25 mM L-Glutathione reduced) followed by two buffer exchanges with a third refolding buffer (e.g. containing 20 mM Bis-Tris, pH 6.5, 150 mM NaCl).

Accordingly, in various embodiments, the methods disclosed herein further comprise solubilizing the isolated polypeptide; and refolding the solubilized polypeptide to obtain a polypeptide having protein ligase activity.

After the step of isolating or refolding, the method further comprises purifying the polypeptide by a suitable and well-known method that is not particularly limited.

In various embodiments, after the step of isolating, the isolated polypeptides obtained as a result of the culturing and isolating steps can be subsequently purified by known methods of separation of various types using the physical or chemical properties of the polypeptide. Specific examples may include treatment using a standard protein precipitating agent, ultrafiltration, various types of liquid chromatography such as molecular sieve chromatography (gel filtration), absorption chromatography, ion exchange chromatography or affinity chromatography, a dialysis method, and a combination thereof.

In various embodiments, after the step of refolding, the refolded polypeptide can be subsequently purified from the refolding buffer by well-known methods alone or in combination, including but not limited to ammonium sulfate precipitation or ethanol precipitation, acid extraction, anion or cation exchange chromatography, phosphocellulose chromatography, hydrophobic interaction, Chromatography, affinity chromatography, hydroxyapatite chromatography, and lectin chromatography. In various embodiments, the polypeptide is purified by affinity chromatography and size exclusion chromatography.

In various embodiments, the purified polypeptide is constitutively active, more particularly the purified polypeptide is stable and constitutively active exhibiting protein ligase activity.

In various embodiments, the method does not comprise an activation step or use of an activating agent for enzymatically activating the protein ligase activity of the polypeptide after the purification step, more particularly the method does not comprise an acid-activation step in producing a stable constitutively active polypeptide exhibiting protein ligase activity. Accordingly, the method disclosed herein produces a polypeptide having protein ligase activity, more particularly a constitutively active protein ligase, with the proviso that the method does not comprise an acid-activation step (i.e. a low pH activation step). In the context of the acidic activation step, the low pH refers to a pH value less than 7, equal or less than 6.5, equal or less than 5, equal or less than 4.5, preferably equal or less than 4.

Accordingly, in various embodiments, the method of producing the polypeptide as disclosed herein, comprises: culturing a host cell comprising a nucleic acid molecule encoding the polypeptide under conditions that allows expression of the polypeptide, isolating the polypeptide from the host cell or culture medium, and purifying the polypeptide to obtain the polypeptide having protein ligase activity. The method optionally further comprises solubilizing and refolding the isolated polypeptide.

In various embodiments, the method of producing the polypeptide as disclosed herein, comprises (a) culturing a host cell comprising a nucleic acid molecule encoding the polypeptide disclosed herein under conditions that allows expression of the polypeptide, (b) isolating said expressed polypeptide from the host cell or culture medium, which may optionally comprise cell lysis of the host cell to extract inclusion bodies of the polypeptide; (c) denaturing of the polypeptide using a solubilizing buffer containing a denaturant; (d) diluting the denaturant using a dilution buffer (i.e. first refolding buffer); (e) removing the denaturant through a dialysis method using one or more dialysis buffers (i.e. second and third refolding buffers) to obtain a refolded polypeptide; and (f) purifying the refolded polypeptide. The purified folded polypeptide can be stored until use and is a stable constitutively active polypeptide exhibiting protein ligase activity.

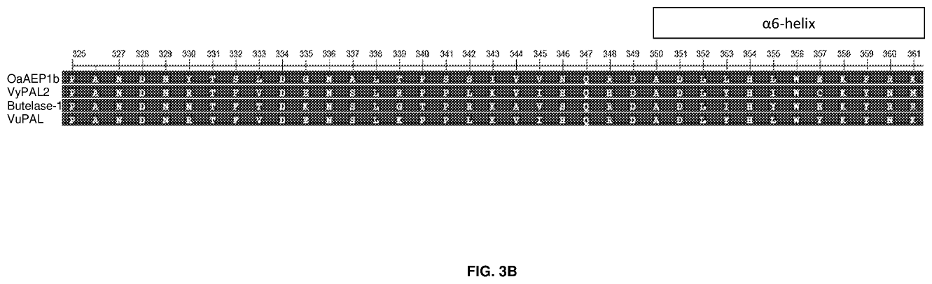

As illustrated in FIG. 4, in various embodiments, the method of producing the polypeptide as disclosed herein, comprises:
a) culturing an E. coli cell disclosed herein in an LB media, where expression of the polypeptide disclosed herein as inclusion bodies is induced by IPTG;
b) lysing the host cells using a lysis buffer and forming cell pellets containing the polypeptide;
c) solubilizing the polypeptide in a solubilizing buffer containing urea to provide a solubilized and denatured polypeptide;
d) diluting the urea in (c) using a dilution buffer;
e) refolding the solubilized polypeptide by removing the urea from (d) with a dialysis method using two or more dialysis buffers to provide a folded polypeptide with protein ligase activity; and
f) purifying the folded polypeptide with affinity chromatography followed by size exclusion chromatography.

It will be appreciated that the polypeptides described herein and produced via the methods disclosed herein may be used for protein ligation, in particular for cyclizing one or more peptide(s).

In particular, two or more peptides may be ligated by the polypeptides disclosed herein. This may include formation of macrocycles consisting of two or more peptides, preferable are macrocyclic dimers. The peptides to be ligated can be any peptides, as long as at least one of them contains a recognition and ligation sequence that is recognized, bound and ligated by the ligase/cyclase. Suitable peptides have been described above in connection with the cyclization strategy. The same peptides can also be used for ligation to another peptide that may be the same or different. One of the peptides to be ligated may for example be a polypeptide that has enzymatic activity or another biological function. The peptides to be ligated may also include marker peptides or peptides that comprise a detectable marker, such as a fluorescent marker or biotin.

Accordingly, there is provided a method for cyclizing a peptide, polypeptide or protein, the method comprising incubating said peptide, polypeptide, or protein with the polypeptides disclosed herein having ligase/cyclase activity under conditions that allow cyclization of said peptide.

Accordingly, there is provided a method for ligating at least two peptides, polypeptides or proteins, the method comprising incubating said peptides, polypeptides or proteins with the polypeptides disclosed herein under conditions that allow ligation of said peptides.

The invention is further illustrated by the following non-limiting examples and the appended claims.

### EXAMPLES

### Materials and Methods

Design and expression of constitutively active OaAEP1b-C247A-4351: The expression constructs spanning residues Gly55 to Asp351, with an N-terminal hexahistidine tag followed by a TEV cleavage site, were synthesized by BioBasic (Singapore). These constructs were expressed in E. coli BL21 (T1R) cells and cultivated at 37° C. to an OD₆₀₀˜1 in LB media (Biobasic, Singapore). The proteins were overexpressed following induction with 0.5 mM IPTG at 18° C. for 18 h. Cells were pelleted and stored at −80° C. before purification.

Refolding and purification of constitutively active OaAEP1b-C247A-4351 using dialysis method: With all steps performed at 4° C., protein purification was achieved by resuspending thawed pellets in 30 mL of lysis buffer (100 mM Bis-Tris, pH 6.5, 500 mM NaCl, 10% (v/v) glycerol), sonicating the pellets followed by clearing the lysates by centrifugation at 58,000 g for 45 min. The insoluble fraction was resolubilized in 10 mL of resolubilizing buffer (50 mM Bis-Tris, pH 6.5, 150 mM NaCl, 1 mM EDTA, 50 mM Glycine, 8 M urea) overnight, with agitation. The concentration of denatured protein was determined using nanodrop and diluted to ˜2 mg mL-1 with buffer 1 (50 mM Bis-Tris, pH 6.5, 150 mM NaCl, 500 mM L-Arginine, 1 M urea, 0.25 mM L-Glutathione oxidized, 2.5 mM L-Glutathione reduced). The diluted denatured protein was dialyzed against buffer 2 (50 mM Bis-Tris, pH 6.5, 150 mM NaCl, 200 mM L-Arginine, 0.125 mM L-Glutathione oxidized, 1.25 mM L-Glutathione reduced) for 8 hr, followed by two buffer exchanges with buffer 3 (20 mM Bis-Tris, pH 6.5, 150 mM NaCl), for a duration of 8 hr for each buffer exchange. The refolded proteins were purified by affinity chromatography (HisTrap column, Cytiva), followed by size exclusion chromatography (HiLoad 16/600 Superdex 200, Cytiva). The proteins eluted as monomers during size exclusion purification. OaAEP1b-C247A-Δ351 was concentrated to 1 mg mL-1 in 20 mM Bis-Tris, pH 6.5, 150 mM NaCl, 5% (v/v) glycerol using centrifugation and concentrators with a 10 kDa cut-off (Amicon, USA). Aliquots were flash-frozen in liquid nitrogen and stored at −80° C. until use.

Protein samples were collected after resolubilization, dialysis and purification and were analyzed with SDS-PAGE. Western blot analysis was also carried out using anti-His antibody obtained from Sigma (catalog number: SAB4301134) to validate the purification of the protein.

Purification and expression of full length OaAEP1b-C247A: The full-length OaAEP1b-C247A construct was synthesized by BioBasic and was expressed in E. coli BL21 (T1R) cells. Expression and activation of OaAEP1b-C247A were done according to reference [17].

Peptide cyclization assay: The peptide used for cyclization assay was purchased commercially from GenScript, NH₂-GLPVSTKPVATRNAL-COOH (SEQ ID NO:17). Cyclization assays were performed in 50 μl reaction mixtures containing 20 mM phosphate buffer, pH 6.5, ligases (40 nM) and peptide substrates (20 μM). Reaction was performed at 37° C., for 1 hour. The cyclization product was analyzed by MALDI-TOF MS (ABI 4800 MALDI TOF/TOF).

Kinetics assay: The kinetic properties of the peptide ligation of the constitutively active PAL were studied using a FRET assay. Two peptides synthesized by GenScript: PIE {EDANS} YNAL (SEQ ID NO:18) and GIK {DABSYL} SIP (SEQ ID NO:19) were mixed at a molar ratio of 1:3. Upon ligation, the peptide PIE {EDANS} YNGIK {DABSYL} SIP (SEQ ID NO: 20) is produced. 50 nM of PAL enzyme is mixed with various concentrations of the peptide mixture. The EDANS fluorescence signal was measured with an excitation wavelength of 336 nm and an emission wavelength of 490 nm. A reduction in EDANS fluorescence signal occurs upon ligation of the two peptides due to quenching by DABSYL. The variation in fluorescence signal for each substrate mixture concentration was measured after addition of the enzyme to initiate the reaction. The rate of decrease in fluorescence signal during the first 30 seconds after enzyme addition was plotted against the substrate concentration to obtain the Vₘₐₓ, k_{cat} and K_{M} values for each enzyme.

Active site titration: The procedure described in reference was followed. The enzyme preparation was diluted to a concentration of 280 nM using a buffer containing 20 mM sodium phosphate at pH 6.5, 5 mM 2-Mercaptoethanol. Solutions containing serial twofold dilution of inhibitor YVAD-cmk were prepared in a black microtiter plate (Greiner Bio-One) using buffer as diluent. The enzyme was subsequently added to the wells containing the inhibitor to a final volume of 50 μL. The plate was incubated for 1 hour at room temperature before adding FRET peptides (PIE {EDANS} YNAL (SEQ ID NO:18) and GIK {DABSYL} SIP (SEQ ID NO: 19)), which were mixed at a molar ratio of 1:3 giving a final enzyme: substrate molar ratio of 1:200. The EDANS fluorescence signal was measured with an excitation wavelength of 336 nm and an emission wavelength of 490 nm. Relative fluorescence units (RFU) of quenched EDANS signal were plotted against time. The value of the initial velocity (Vi) was determined from the slope of the RFU (t) curve. The measured value of Vi was subsequently normalized by dividing with the initial rate obtained in the absence of inhibitor (control V₀). The calculated Vi/V0 ratio was plotted against inhibitor concentrations, generating an inhibition curve. The titre of the enzyme active site was then inferred from the intercept of this inhibition curve with the x-axis, assuming a 1:1 interaction between enzyme and inhibitor, which is in agreement with experimental crystallographic structures of homologous PALs with a peptide substrate published previously [34, 36].

Conjugation of TrmJ: A concentration of 200 nM of OaAEP1b-C247A-4351 was used to conjugate 10 μM of TrmJ-NAL with 50 μM of a short fluorescence peptide synthesized by Genscript: GIGGIYRK-FITC (SEQ ID NO:21). This reaction was carried out in a 20 mM NaH₂PO₄, pH 6.5 at 37° C. for 1 hour with a final volume of 500 μL. A volume of 50 UL of the reaction was mixed with 5×SDS loading dye after 5, 10, 20, 30 and 60 minutes. The amount of conjugated TrmJ-NAL at all time points was then analyzed using SDS PAGE.

```
TABLE 1
List of SEQ ID NOs and amino acid sequences described herein.
Boxed sequences indicate the linker sequence. Underlined sequences
indicating the cap domain. Black highlighted sequence indicating
  the first N-terminal helix of the cap domain (e.g. α6-helix).
  Bold letters indicate the given or corresponding amino acid
    residue at position 351 according to SEQ ID NO: 1.
```

| SEQ ID | Name/ | |
|---|---|---|
| NO: | Description | Amino Acid Sequence |
| 1 | Wilt-type OaAEP1b Source: Oldenlandia affinis | 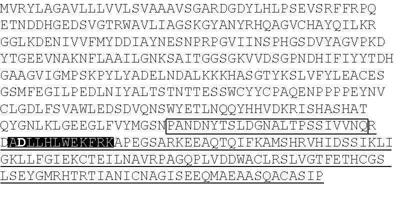 |
| 2 | OaAEP1b-C247A Source: Oldenlandia affinis | 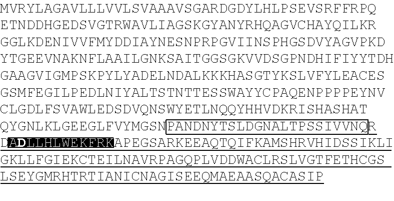 |
| 3 | Core domain + Linker + Cap domain of OaAEP1b-C247A Source: Oldenlandia affinis | 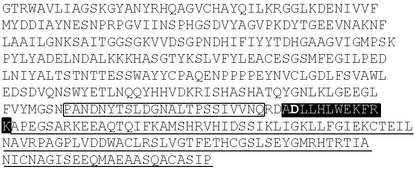 |
| 4 | OaAEP1b- C247A-Δ351 (construct for recombinant expression) | 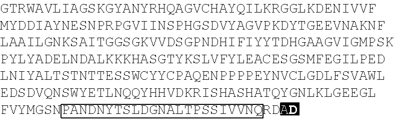 |
| 5 | His-tag and TEV | MHHHHHHSSGVDLGTENLYFQSV |
| | cleavage | |
| | sequence | |
| 6 | OaAEP1b- C247A-Δ351 with His-tag and TEV cleavage sequence (contruct for recombinant expression) | 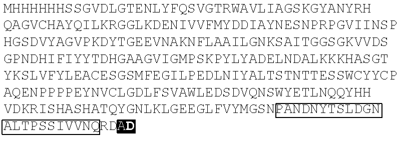 |
| 7 | VyPAL2 Source: Viola yedoensis | 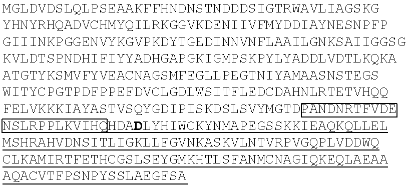 |
| 8 | Core domain + Linker + Cap domain of VyPAL2 Source: Viola yedoensis | 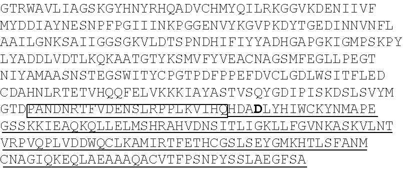 |
| 9 | Butelase-1 Source: Clitoria ternatea | 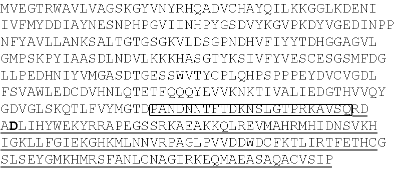 |
| 10 | Core domain + Linker + Cap domain of Butelase-1 Source: Clitoria ternatea | 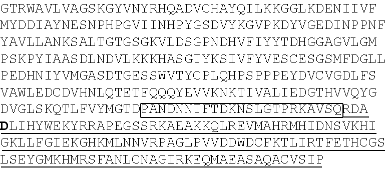 |
| 11 | Butelase-2 Source: Clitoria ternatea | 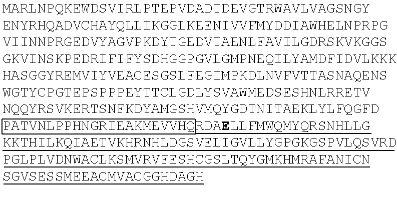 |
| 12 | Core domain + Linker + Cap domain of Butelase-2 Source: Clitoria ternatea | 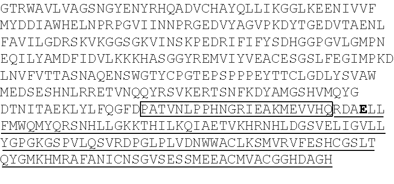 |
| 13 | VcPAL Source: Viola canadensis | 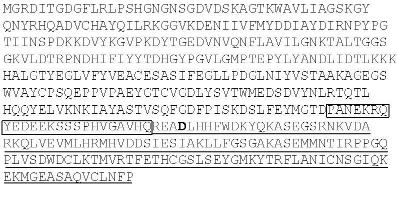 |
| 14 | Core domain + Linker + Cap domain of VcPAL Source: Viola canadensis | 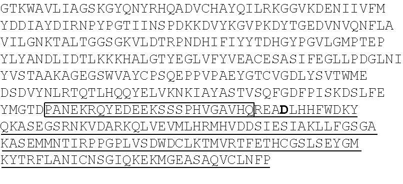 |
| 15 | VuPAL Source: Viola Uliginosa | 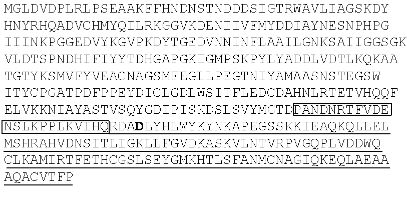 |
| 16 | Core domain + Linker + Cap domain of VuPAL Source: Viola Uliginosa | 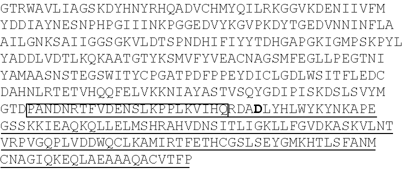 |
| 17 | Peptide substrate | GLPVSTKPVATRNAL |
| | (LS) for ligation | |
| 18 | Peptide A | PIEYNAL |
| 19 | Peptide B | GIKSIP |
| 20 | Ligated peptide | PIEYNGIKSIP |
| | (A + B) | |
| 21 | fluorescence | GIGGIYRK |
| | peptide | |

### Results and Discussion

### Example 1: Analysis of the Interface Between the Core and Cap Domains of OaAEP1b

The crystal structure of OaAEP1b (PDB access code: 5H01) allows a precise analysis of the set of interactions established between the cap and the core domains in the zymogen form (FIG. 1A). In the context of the plant cells, the cap domain appears to regulate the activity of PALs and AEPs to prevent undesired protein processing or protein/peptide ligation. Four residues, Val344-Val345-Asn346-Gln347 preceding the α6 helix (the first N-terminal helix of the cap domain), are located at the interface between the cap and core domain. In particular, Gln347 penetrates deeply into the S1 pocket establishing several polar interactions with surrounding active site residues [17]. The interface between the cap and the core domain extends over a total surface of 1,227 Å2 and involves 41 residues of the core domain which make contacts with 31 residues from the cap domain. A total of nine hydrogen bonds and fourteen salt bridges are formed between residues from the cap and the core domain and the estimated total binding energy for this interaction is-18.8 kcal/mol at neutral pH, as measured by PISA (https://www.ebi.ac.uk/pdbe/pisa/). Of note, seven Glu residues are found in the interface between the cap and the core domain of OaAEP1b (FIG. 1A). Separation of the two domains requires acidification of the milieu to pH values ranging between 4.0 and 4.5 with addition of non-ionic detergents such as N-Laurylsarcosine. At these pH values, Glu residues are no longer negatively charged, disrupting the favourable electrostatic interactions between the two domains, and favouring proteolytic cleavage in trans (FIG. 1B).

### Example 2: Design and Expression of a Constitutively Active OaAEP1b-C247A

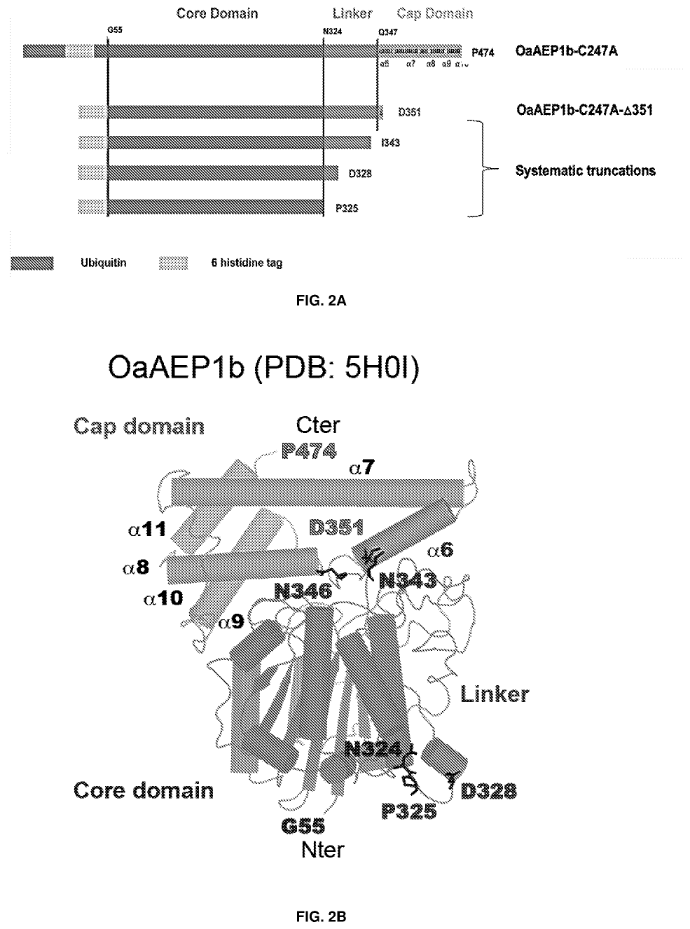

From the analysis on OaAEP1b and from other AEPs and PAL crystal structures, it appears that the α6-helix and the four residues Val344-Val345-Asn346-Gln347 immediately preceding this a-helix, must play an important role in stabilizing the enzyme in its zymogen form. Therefore, a series of truncated constructs were designed targeting residues located in the α6-helix region and in the linker between the cap and core domain of OaAEP1b-C247A (FIG. 2A).

All four constructs were expressed in E. coli BL21 T1R and designed to include the core domain of OaAEP1b-C247A (residues Gly55 to Asn324 according to numbering of SEQ ID NO: 1), discarding the signal peptide region (residues 1-54) [17]. In addition to this core region necessary for activity, the four constructs designed included incremental sections from the linker and α6 helix encompassing putative acid-activation sites located after Asn or Asp residues, such as Asp328 or Asn336 (FIGS. 2B and 3A). All four constructs showed robust levels of expression in E. coli although the corresponding proteins were all expressed as inclusion bodies. Next, extraction of the proteins was attempted from the insoluble fraction by urea solubilization followed by refolding. Out of the four OaAEP1b-C247A constructs tested, only the OaAEP1b-C247A-Δ351 protein could be refolded. For the other three truncated proteins tested, severe precipitation during the refolding procedure was observed, indicating that segments in the region spanning residues Pro325-Asp351 are required for protein solubility.

### Example 3: Refolding and Purification of OaAEP1b-C247a-Δ351

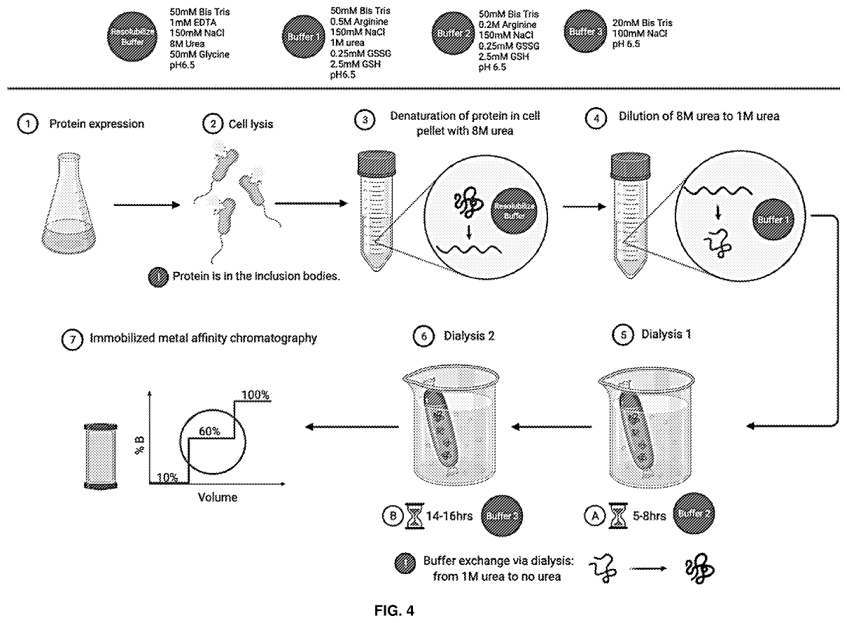

The expression of OaAEP1b-C247A-Δ351 was observed to be of a good level in E. coli inclusion bodies (FIG. 5A). Thus, the inclusion bodies were first resolubilized in 8 M urea. The protein was subsequently refolded via stepwise dilution and reduction of urea concentration from 8 M to 0 M using buffer 1 and buffer 2, respectively (see methods and FIG. 4). After stepwise dialysis, a two-step purification of the refolded OaAEP1b-C247A-Δ351 was carried out. First, metal affinity chromatography (HisTrap column, Cytiva) was used followed by size exclusion chromatography (Superdex 200 16/600 pg, Cytiva) (FIGS. 5B and 5C). These steps led to a pure monomeric fraction of OaAEP1b-C247A-Δ351 (FIG. 5C). A yield of 1.75 mg/100 mL of bacterial culture of purified OaAEP1b-C247A-Δ351 was routinely able to be obtained.

### Example 4: Cyclization Activity of OaAEP1b-C247a-Δ351

To evaluate the cyclization activity of the purified OaAEP1b-C247A-Δ351, the enzyme was tested against a linear NH2-GLPVSTKPVATRNAL-COOH (SEQ ID NO:17) peptide substrate (labelled “LS”) (FIG. 6A). The cyclization reaction was performed at 37° C., and samples were collected every two minutes. MALDI-TOF MS was subsequently utilized to detect a cyclized product (labelled “CP”). Successful cyclization carried out by the active ligase of the LS with a mass of 1524 Da would result in CP with a mass of 1321 Da (FIG. 6A). After twelve minutes of reaction time, OaAEP1b-C247A-Δ351 had converted the majority of the linear substrate to the circularized product. No LS peak could be detected compared to a high CP peak detected in the MALDI-TOF mass spectra of the reaction mixture. (FIG. 6B), indicating a complete cyclization reaction of the substrate by OaAEP1b-C247A-Δ351.

### Example 5: Comparison of Ligase Activity of Constitutively Active Vs Acid-Activated PAL

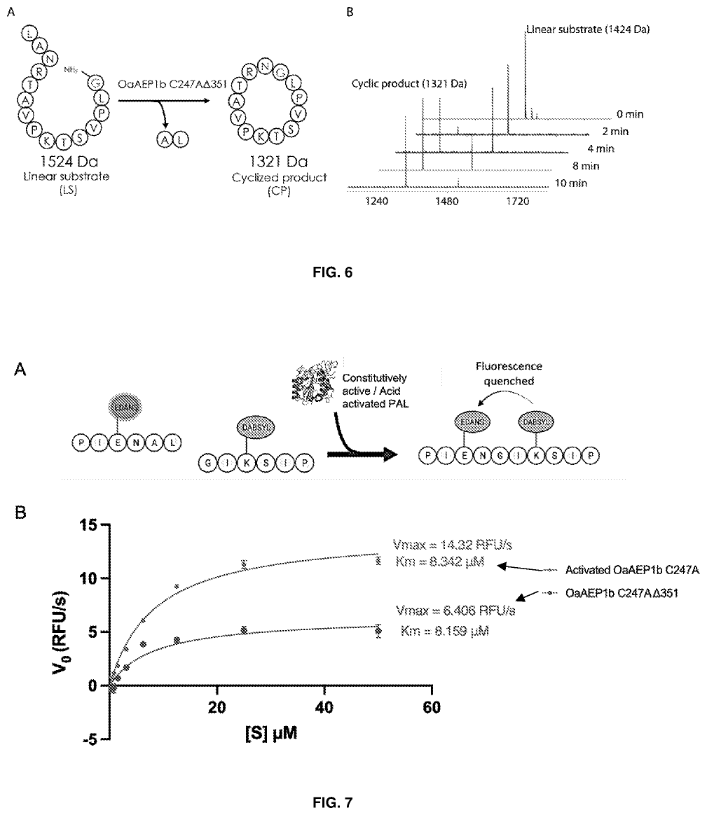

Next, using a FRET ligation assay, the ligase activity of the truncated OaAEP1b-C247A-Δ351 was compared with its acid-activated zymogen counterpart. Briefly, 50 nM of either enzyme was added to a mixture of two peptides A: PIE (EDANS) YNAL (SEQ ID NO:18) and B: GIK (DABSYL) SIP (SEQ ID NO:19). These two peptides were mixed in a A:B molar ratio of 1:3. Upon ligation, the fluorescence signal emission of the EDANS moiety of A (λₑₘ=490 nm) becomes quenched by the DABSYL moiety of B. (FIG. 7A). This assay the ligation rate to be followed between both peptides in real-time, giving access to the kinetic parameters of the truncated enzyme. It was observed that the truncated purified protein has a ligation activity comparable (about 2-fold less) to its acid-activated zymogen counterpart and previously reported OaAEP1b-C247A [17]. The Vmax and Km values are 6.40 RFU/see and 8.16 uM, respectively, for OaAEP1b-C247A-Δ351 compared with 14.32 RFU/see and 8.34 M for acid-activated OaAEP1b-C247A Vmax and Km values, respectively.

As the constitutively active PAL was obtained using a refolding protocol, the exact final proportion of OaAEP1b-C247A-4351 proteins adopting an active conformation is not known, giving some uncertainty on the determination of the kinetic parameters. Thus, in order to refine the comparison of the activity of the refolded enzyme with the acid-activated one, the titration of their active sites was performed following the procedure outlined in [33].

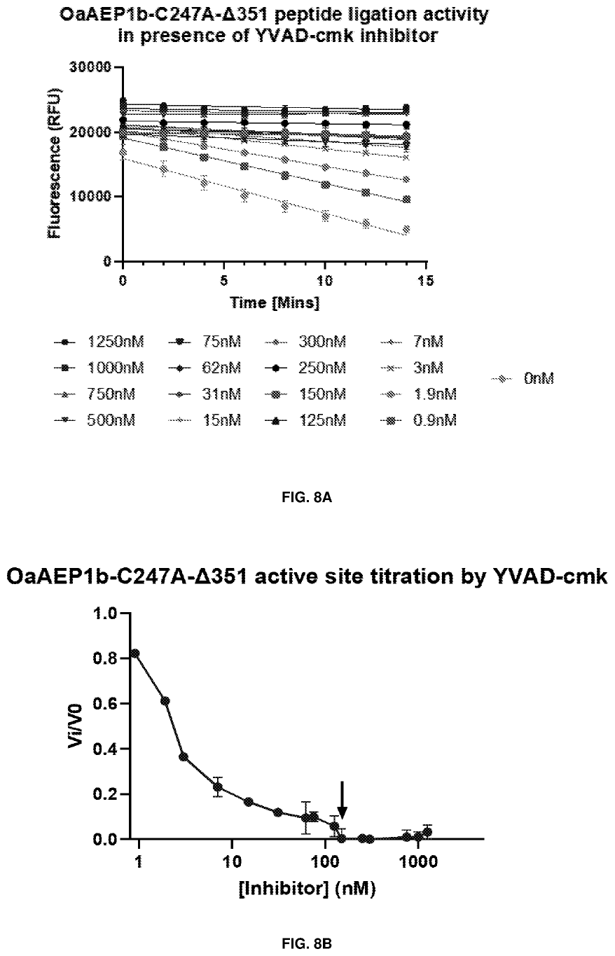

To understand the difference in the Vmax between OaAEP1b-C247A-Δ351 and acid-activated OaAEP1b-C247A an active site titration was performed of OaAEP1b-C247A-Δ351 using a FRET ligation assay, after a 1 hour incubation with varying concentrations of a covalent AEP inhibitor, Ac-YVAD-cmk [34]. The result of the active site titration showed that about 53% of the measured protein concentration is active and amenable to complete inhibition (FIG. 8). Remarkably, this difference in the concentration of active protein matches the measured difference in Vmax and suggests that the activity of the OaAEP1b-C247A-Δ351 is very similar to the activity of the acid-activated OaAEP1b-C247A.

### Example 6: Conjugation of the tRNA Methyltransferase, TrmJ with a Fluorescent Peptide

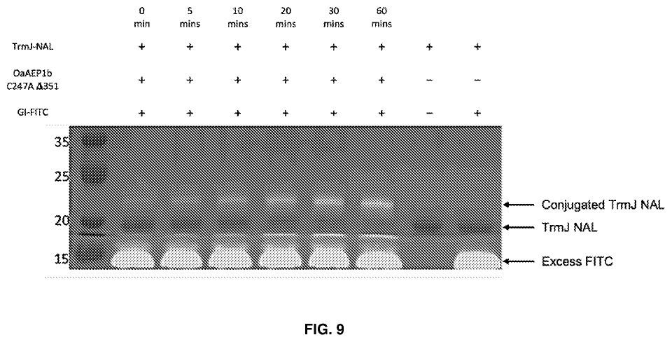

To evaluate OaAEP1b-C247A-Δ351 conjugation capability, a protein of 20 kDa, tRNA methyltransferase, TrmJ were conjugated [32]. TrmJ was modified to include the C-terminal OaAEP1b-C247A-Δ351 preferred tripeptide recognition motif (Asn-Ala-Leu). Using 200 nM of OaAEP1b-C247A-Δ351, TrmJ present in the solution was able to conjugate with a short fluorescence peptide consisting of an N-terminal Gly/Ile (GIGGIYRK-FITC) (SEQ ID NO: 21). The conjugation rate at 37° C. was analyzed using SDS PAGE at six different time points. An increment of the FITC signal was observed at every time point, and after an hour of reaction time, most of TrmJ was labelled with FITC (FIG. 9). These results demonstrated that the constitutively active OaAEP1b-C247A A351 can efficiently conjugate a protein.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, kits and uses described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

### REFERENCES

[1] Bagert J D & Muir T W (2021) Molecular Epigenetics: Chemical Biology Tools Come of Age. Annu Rev Biochem 90, 287-320.
[2] Schmidt M, Toplak A, Quaedflieg P J & Nuijens T (2017) Enzyme-mediated ligation technologies for peptides and proteins. Curr Opin Chem Biol 38, 1-7.
[3] Cao Y, Nguyen G K T, Tam J P & Liu C-F (2015) Butelase-mediated synthesis of protein thioesters and its application for tandem chemoenzymatic ligation. Chem Commun 51, 17289-17292.
[4] Bi X, Yin J, Nguyen G K T, Rao C, Halim N B A, Hemu X, Tam J P & Liu C F (2017) Enzymatic Engineering of Live Bacterial Cell Surfaces Using Butelase 1. Angew Chemie—Int Ed 56, 7822-7825.
[5] Kwon S, Duarte J N, Li Z, Ling J J, Cheneval O, Durek T, Schroeder C I, Craik D J & Ploegh H L (2018) Targeted Delivery of Cyclotides via Conjugation to a Nanobody. ACS Chem Biol 13, 2973-2980.
[6] Cao Y, Nguyen G K T, Chuah S, Tam J P & Liu C-F (2016) Butelase-Mediated Ligation as an Efficient Bioconjugation Method for the Synthesis of Peptide Dendrimers. Bioconjug Chem 27, 2592-2596.
[7] Nguyen G K T, Cao Y, Wang W, Liu C F & Tam J P (2015) Site-Specific N-Terminal Labeling of Peptides and Proteins using Butelase 1 and Thiodepsipeptide. Angew Chemie Int Ed 54, 15694-15698.
[8] Nguyen G K T, Hemu X, Quek J P & Tam J P (2016) Butelase-Mediated Macrocyclization of d-Amino-Acid-Containing Peptides. Angew Chemie-Int Ed 55, 12802-12806.
[9] Nguyen G K T, Kam A, Loo S, Jansson A E, Pan L X & Tam J P (2015) Butelase 1: A Versatile Ligase for Peptide and Protein Macrocyclization. J Am Chem Soc 137, 15398-15401.
[10] Cao Y, Nguyen G K T, Qiu Y, Liu C-F, Tam J P & Hemu X (2016) Butelase-mediated cyclization and ligation of peptides and proteins. Nat Protoc 11, 1977-1988.
[11] Nguyen G K T, Wang S, Qiu Y, Hemu X, Lian Y & Tam J P (2014) Butelase 1 is an Asx-specific ligase enabling peptide macrocyclization and synthesis. 10.
[12] Mao H, Hart S A, Schink A & Pollok B A (2004) Sortase-Mediated Protein Ligation: A New Method for Protein Engineering. J Am Chem Soc 126, 2670-2671.
[13] Jackson M A, Nguyen L T T, Gilding E K, Durek T & Craik D J (2020) Make it or break it: Plant AEPs on stage in biotechnology. Biotechnol Adv 45, 107651.
[14] James A M, Haywood J & Mylne J S (2018) Macrocyclization by asparaginyl endopeptidases. New Phytol 218, 923-928.
[15] Hemu X, Sahili A El, Hu S, Wong K, Chen Y, Wong Y H, Zhang X, Serra A, Goh B C, Darwis D A, Chen M W, Sze S K, Liu C F, Lescar J & Tam J P (2019) Structural determinants for peptide-bond formation by asparaginyl ligases. Proc Natl Acad Sci USA 116, 11737-11746.
[16] Harris K S, Durek T, Kaas Q, Poth A G, Gilding E K, Conlan B F, Saska I, Daly N L, Van Der Weerden N L, Craik D J & Anderson M A (2015) Efficient backbone cyclization of linear peptides by a recombinant asparaginyl endopeptidase. Nat Commun 6.
[17] Yang R, Wong Y H, Nguyen G K T T, Tam J P, Lescar J & Wu B (2017) Engineering a Catalytically Efficient Recombinant Protein Ligase. J Am Chem Soc 139, 5351-5358.
[18] Dall E & Brandstetter H (2013) Mechanistic and structural studies on legumain explain its zymogenicity, distinct activation pathways, and regulation. Proc Natl Acad Sci USA 110, 10940-10945.
[19] Dall E, Zauner F B, Soh W T, Demir F, Dahms S O, Cabrele C, Huesgen P F & Brandstetter H (2020) Structural and functional studies of Arabidopsis thaliana legumain beta reveal isoform specific mechanisms of activation and substrate recognition. J Biol Chem 295, 13047-13064.
[20] Bernath-Levin K, Nelson C, Elliott A G, Jayasena A S, Millar A H, Craik D J & Mylne J S (2015) Peptide macrocyclization by a bifunctional endoprotease. Chem Biol 22, 571-582.
[21] Zauner F B, Elsässer B, Dall E, Cabrele C & Brandstetter H (2018) Structural analyses of Arabidopsis thaliana legumain reveal differential recognition and processing of proteolysis and ligation substrates. J Biol Chem 293, 8934-8946.
[22] Zauner F B, Dall E, Regl C, Grassi L, Huber C G, Cabrele C & Brandstetter H (2018) Crystal structure of plant legumain reveals a unique two-chain state with pH-dependent activity regulation. Plant Cell 30, 686-699.
[23] James A M, Haywood J, Leroux J, Ignasiak K, Elliott A G, Schmidberger J W, Fisher M F, Nonis S G, Fenske R, Bond C S & Mylne J S (2019) The macrocyclizing protease butelase 1 remains autocatalytic and reveals the structural basis for ligase activity. Plant J 98, 988-999.
[24] Hemu X, Sahili A El, Hu S, Zhang X, Serra A, Goh B C, Darwis D A, Chen M W, Sze S K, Liu C, Lescar J & Tam J P (2020) Turning an Asparaginyl Endopeptidase into a Peptide Ligase.
[25] Dall E, Stanojlovic V, Demir F, Briza P, Dahms S O, Huesgen P F, Cabrele C & Brandstetter H (2021) The Peptide Ligase Activity of Human Legumain Depends on Fold Stabilization and Balanced Substrate Affinities.
[26] Haywood J, Schmidberger J W, James A M, Nonis S G, Sukhoverkov K V., Elias M, Bond C S & Mylne J S (2018) Structural basis of ribosomal peptide macrocyclization in plants. Elife 7.
[27] Zhao L, Hua T, Crowley C, Ru H, Ni X, Shaw N, Jiao L, Ding W, Qu L, Hung L W, Huang W, Liu L, Ye K, Ouyang S, Cheng G & Liu Z J (2014) Structural analysis of asparaginyl endopeptidase reveals the activation mechanism and a reversible intermediate maturation stage. Cell Res 24, 344-358.
[28] Jackson M A, Gilding E K, Shafee T, Harris K S, Kaas Q, Poon S, Yap K, Jia H, Guarino R, Chan L Y, Durek T, Anderson M A & Craik D J (2018) Molecular basis for the production of cyclic peptides by plant asparaginyl endopeptidases. Nat Commun 9, 1-12.
[29] Kuroyanagi M, Nishimura M & Hara-Nishimura I (2002) Activation of Arabidopsis Vacuolar Processing Enzyme by Self-Catalytic Removal of an Auto-Inhibitory Domain of the C-Terminal Propeptide. Plant Cell Physiol 43, 143-151.
[30] Mulvenna J P, Mylne J S, Bharathi R, Burton R A, Shirley N J, Fincher G B, Anderson M A & Craik D J (2006) Discovery of cyclotide-like protein sequences in graminaceous crop plants: Ancestral precursors of circular proteins? Plant Cell 18, 2134-2144.
[31] Mylne J S, Chan L Y, Chanson A H, Daly N L, Schaefer H, Bailey T L, Nguyencong P, Cascales L & Craik D J (2012) Cyclic peptides arising by evolutionary parallelism via asparaginyl-endopeptidase-mediated biosynthesis. Plant Cell 24, 2765-2778.
[32] Jaroensuk J, Atichartpongkul S, Chionh Y H, Hwa Wong Y, Liew C W, McBee M E, Thongdee N, Prestwich E G, DeMott M S, Mongkolsuk S, Dedon P C, Lescar J & Fuangthong M (2016) Methylation at position 32 of tRNA catalyzed by TrmJ alters oxidative stress response in Pseudomonas aeruginosa. Nucleic Acids Res 44, 10834-10848.
[33] Harris K S, Guarino R F, Dissanayake R S, Quimbar P, McCorkelle O C, Poon S, Kaas Q, Durek T, Gilding E K, Jackson M A, Craik D J, van der Weerden N L, Anders R F & Anderson M A (2019) A suite of kinetically superior AEP ligases can cyclise an intrinsically disordered protein. Sci Rep 9, 1-13.
[34] Dall, E., Zauner, F. B., Soh, W. T., Demir, F., Dahms, S. O., Cabrele, C., Huesgen, P. F., and Brandstetter, H. (2020). Structural and functional studies of Arabidopsis thaliana legumain beta reveal isoform specific mechanisms of activation and substrate recognition. Journal of Biological Chemistry 295:13047-13064.
[35] Tang T M S, Cardella D, Lander A J, Li X, Escudero J S, Tsai Y H & Luk L Y P (2020) Use of an asparaginyl endopeptidase for chemo-enzymatic peptide and protein labeling. Chem Sci 11, 5881-5888.
[36] Hu S, El Sahili A, Kishore S, Wong Y H, Hemu X, Goh B C, Wang Z, Tam J P, Liu C-F & Lescar J (2022) Structural basis for proenzyme maturation, substrate recognition and ligation by a hyperactive 10.1093/plcell/koac281 peptide asparaginyl ligase Plant Cell Sep 13:koac281. doi: 10.1093/plcell/koac281

1. A polypeptide having protein ligase activity, comprising:
i. an amino acid sequence as set forth in SEQ ID NO: 3 (OaAEP1b C247A core domain+linker+cap domain);
ii. an amino acid sequence that shares at least 55, preferably at least 60, preferably at least 70, more preferably at least 80, most preferably at least 90% sequence identity with the amino acid sequence of (i) over its entire length; or
iii. an amino acid sequence that shares at least 70, preferably at least 80, more preferably at least 90, most preferably at least 95% sequence homology with the amino acid sequence of (i) over its entire length,
wherein the amino acid sequence of (i)-(iii) comprises a C-terminal truncation after amino acid position 351, wherein position numbering is in accordance with SEQ ID NO:1 (OaAEP1b).

2. The polypeptide of claim 1, wherein the polypeptide comprises amino acid residue D at the positions corresponding to positions 349 and 351 of SEQ ID NO: 1.

3. The polypeptide of claim 1, wherein the polypeptide comprises:
a) amino acid residue A at the position corresponding to position 350 of SEQ ID NO:1; and/or
b) amino acid residue R or H at the position corresponding to position 348 of SEQ ID NO:1; and/or
c) amino acid residue Q at the position corresponding to position 347 of SEQ ID NO:1.

4. The polypeptide of claim 1, wherein the polypeptide comprises:
a) amino acid residue A or V at the position corresponding to position 344 of SEQ ID NO:1; and/or
b) amino acid residue V or I at the position corresponding to position 345 of SEQ ID NO:1; and/or
c) amino acid residue V, N, H or S at the position corresponding to position 346 of SEQ ID NO:1; and/or
d) amino acid residue D at the position corresponding to position 351 of SEQ ID NO:1.

5. The polypeptide of claim 1, wherein the polypeptide comprises the amino acid sequence set forth in SEQ ID NO:3 (OaAEP1b C247A core domain+linker+cap domain) comprising a C-terminal truncation after amino acid position 351.

6. The polypeptide of claim 1, wherein the C-terminal truncation starts at an amino acid position within the first N-terminal helix of the cap domain of the amino acid sequence.

7. The polypeptide of claim 1, wherein the polypeptide comprises an amino acid sequence as set forth in SEQ ID NO: 4 (OaAEP1b-C247A-Δ351), wherein the amino acid at position 351 is the C-terminus of the polypeptide.

8. The polypeptide of claim 1, wherein
the polypeptide further comprises a His-tag at the N-terminal of the amino acid sequence.

9. The polypeptide of claim 1, wherein the polypeptide is a constitutively active protein ligase.

10. The polypeptide of claim 1, wherein the polypeptide is a recombinant polypeptide having protein ligase activity.

11. A nucleic acid molecule encoding the polypeptide according to claim 1.

12-15. (canceled)

16. A method for producing a polypeptide having protein ligase activity, comprising:
culturing a host cell comprising a nucleic acid molecule encoding the polypeptide according to claim 1 under conditions that allows expression of the polypeptide;
isolating the polypeptide from the host cell, and
purifying the polypeptide to obtain the polypeptide having protein ligase activity.

17. The method of claim 16, wherein said nucleic acid molecule is comprised in a vector, preferably an expression vector.

18. The method of claim 17, wherein said vector further comprises regulatory elements for controlling expression of said nucleic acid molecule.

19. The method of claim 16, wherein the host cell is a bacteria cell, preferably an E. coli cell, or an insect cell, preferably an Sf9 cell, or a mammalian cell, preferably a Expi293 cell or a ExpiCHO cell.

20. The method of claim 16, wherein the polypeptide having protein ligase activity is constitutively active, optionally wherein the polypeptide is expressed as inclusion bodies.

21. The method of claim 16, wherein the method does not comprise an acid-activation step.

22. The method of claim 16, further comprising lysing the host cell and isolating the expressed polypeptide from the lysed host cell.

23. (canceled)

24. The method of claim 16, further comprising solubilizing the isolated polypeptide.

25. The method of claim 24, further comprising refolding the solubilized polypeptide.
